(19) United States
(12) Patent Application Publication
(10) Pub. No.: US 2008/0171323 A1
(43) Pub. Date: Jul. 17, 2008

(54) Gene Expression Signatures in Blood Leukocytes Permit Differential Diagnosis of Acute Infections

(75) Inventors: Jacques F. Banchereau, Dallas, TX (US); Anna Karolina Palucka, Dallas, TX (US); Octavio Ramilo, Dallas, TX (US); Damien Chaussabel, Richardson, TX (US)

Correspondence Address: CHALKER FLORES, LLP, 2711 LBJ FRWY, Suite 1036, DALLAS, TX, 75234, US

(73) Assignees: BAYLOR RESEARCH INSTITUTE, Dallas, TX (US); BOARD OF REGENTS, THE UNIVERSITY OF TEXAS SYSTEM, Austin, TX (US)

(21) Appl. No.: 11/837,237

(22) Filed: Aug. 10, 2007

Related U.S. Application Data
(60) Provisional application No. 60/837,148, filed on Aug. 11, 2006.

(51) Int. Cl. C12Q 1/68; C40B 30/04; G01N 33/53; G06G 7/48

(52) U.S. Cl. 435/6; 506/9; 435/7.92; 703/11

(57) ABSTRACT

The present invention includes compositions, systems and methods for the early detection and consistent determination of the extent, type and nature of a host immune response and the nature of the infectious disease using gene expression data.

### CROSS-REFERENCE TO RELATED APPLICATIONS

This application claims priority to U.S. Provisional Patent Application Ser. No. 60/837,148, filed Aug. 11, 2006, the entire contents of which are incorporated herein by reference. This application is related to U.S. patent application Ser. No. 11/608,815.

### STATEMENT OF FEDERALLY FUNDED RESEARCH

This invention was made with U.S. Government support under awards by the NIH U19 A1057234-02 and DARPA CA78846. The government has certain rights in this invention.

### TECHNICAL FIELD OF THE INVENTION

The present invention relates in general to the field of diagnostics for infectious diseases, and more particularly, to a system, method and apparatus for the diagnosis, prognosis and tracking of acute and chronic infectious diseases.

### LENGTHY TABLE

The patent application contains a lengthy table section, which includes 11 Supplemental Tables. A copy of the table(s) is(are) available in electronic form from the USPTO web site (http://seqdata.uspto.gov/). An electronic copy of the table will also be available from the USPTO upon request and payment of the fee set forth in 37 CFR 1.19(b)(3).

### BACKGROUND OF THE INVENTION

Without limiting the scope of the invention, its background is described in connection with diagnostic methods for the detection, evaluation, tracking and prognosis of infectious diseases.

Acute infections represent a major cause of mortality in the world [1], especially among children. Concomitantly, the ability to identify infectious agents remains inadequate, particularly if the organism is not present in the blood (or other available tissue). Even if leukocytes are elevated as a result of the infection this will not permit discrimination between gram positive and gram negative bacteria and/or viruses. These diagnostic obstacles might delay initiation of appropriate therapy which can result in unnecessary morbidity and even death [2]. Furthermore, recent outbreaks caused by emerging pathogens [1,3] and the increased risk of biothreat foster the need for improved diagnosis of infectious diseases.

Different classes of pathogens trigger specific pattern-recognition receptors (PRRs) differentially expressed on leukocytes [4,5]. Leukocytes are components of the innate immune system (granulocytes, natural killer cells), the adaptive immune system (T and B lymphocytes), or both (monocytes and dendritic cells). Blood represents both a reservoir and a migration compartment for these cells that might have been exposed to infectious agents, allergens, tumors, transplants or autoimmune reactions. Therefore, blood leukocytes constitute an accessible source of clinically relevant information, and a comprehensive molecular phenotype of these cells can be obtained using gene expression microarrays. Gene expression technology has already brought new perspectives in the diagnosis and prognosis of cancer [6-8], and the analysis of gene expression signatures in blood leukocytes has led to a better understanding of mechanisms of disease onset and responses to treatment [9-11].

### SUMMARY OF THE INVENTION

The present invention includes systems and methods for analyzing samples for the prognosis and diagnosis of infectious diseases using multiple variable gene expression analysis. The gene expression differences that remain can be attributed with a high degree of confidence to the unmatched variation. The gene expression differences thus identified can be used, for example, to diagnose host response to an infectious disease, identify physiological states, identify, track and monitor immune cell activation, design drugs, and monitor therapies.

In one embodiment, the present invention includes a method of identifying the immune response of a human subject predisposed to infectious agents, e.g., viral, bacterial, helminthic, parasitic, fungal, etc., by determining the expression level of a biomarker.

Additional examples of biomarkers include genes related to an infectious agent or disease caused thereby and combinations thereof. The biomarkers may be screened by quantitating the mRNA, protein or both mRNA and protein level of the biomarker. When the biomarker is mRNA level, it may be quantitated by a method selected from polymerase chain reaction, real time polymerase chain reaction, reverse transcriptase polymerase chain reaction, hybridization, probe hybridization, and gene expression array. The screening method may also include detection of polymorphisms in the biomarker. Alternatively, the screening step may be accomplished using at least one technique selected from the group consisting of polymerase chain reaction, heteroduplex analysis, single stand conformational polymorphism analysis, ligase chain reaction, comparative genome hybridization, Southern blotting, Northern blotting, Western blotting, enzyme-linked immunosorbent assay, fluorescent resonance energy-transfer and sequencing. For use with the present invention the sample may be any of a number of immune cells, e.g., leukocytes or sub-components thereof.

Another embodiment includes a method for diagnosing a host response to an infectious disease from a tissue sample, which includes obtaining a gene expression profile from immune tissue sample, wherein expression of the two or more of the following genes is measured, e.g., Supplemental Tables 1 to 11 and combinations thereof. The Lengthy Tables filed concurrently herewith are fully incorporated herein by reference. In one example of the present invention, the gene expression profile or transcriptome value vector may include any of the genes listed in the Tables 1, 4, 5 and Supplementary Tables 1 to 11, and combinations thereof, that form part of the present disclosure, e.g., certain genes may form part of the transcriptome vector(s) that are used to differentiate between genes more highly correlated with an infection with Influenza versus bacteria, e.g., those involved in a response to a virus (e.g., cig5; DNAPTP6; IF127; IF135; IF144; OAS1); an immune response (e.g., BST2; G1P2; LY6E; MX1); anti-apoptosis (e.g., SON); cell growth and/or maintenance (e.g., TRIM14); and miscellaneous genes (e.g., APOBEC3C; Clorf29; FLJ20035; FLJ38348; HSXIAPAF1; KIAA0152; PHACTR2; and USP18). For the differentiation of genes more highly correlated with an infection with a bacteria versus Influenza, it is possible to look at genes involved with translational elongation (e.g., EEF1G); the regulation of translational initiation (e.g., EIF3S5; EIF3S7; EIF4B); protein biosynthesis (e.g., QARS; RPL31; RPL4); the regulation of transcription (e.g., PFDN5); cell adhesion (e.g., CD44); metabolism (e.g., HADHA; PCBP2); and miscellaneous genes, such as dJ507115.1. The tissue used for the source of biomarker, e.g., RNA, may be blood. In one specific embodiment, the gene profiles are obtained and compared between groups of patients, rather than between patients and controls.

Another embodiment includes a method for diagnosing a host response to a specific infectious disease from a tissue sample, which includes obtaining a gene expression profile or transcriptome from an immune tissue sample, wherein expression of the two or more of the following genes may be used to differentiate between an S. aureus infection and an E. coli infection, e.g., signal transduction genes (e.g., CXCL1; JAG1; RGS2); metabolism (e.g., GAPD); PPIB; PSMA7; MMP9; p44S10; protein targeting (e.g., TRAM2); intracellular protein transport (e.g., SEC24C); and miscellaneous genes (e.g., ACTG1; CGI-96; MGC2963; and STAU). Conversely, there may be genes that are most often found to correlate with an E. coli infection and not an S. aureus infection, e.g., intracellular signaling (e.g., RASA1; SNX4); regulation of translational initiation (e.g., AF1Q); regulation of transcription (e.g., SMAD2); cell adhesion (e.g., JUP); metabolism (e.g., PP; MAN1C1); and miscellaneous genes (e.g., FLJ10287; FLJ20152; LRRN3; SGPP1; UBAP2L). The tissue used for the source of biomarker, e.g., RNA, may be blood. The gene profiles are obtained and compared between groups of patients, rather than between patients and controls.

The method of the present invention wherein the step of determining expression levels is performed by measuring amounts of mRNA expressed by the set of genes and/or measuring amounts of protein expressed by the set of genes. The step of determining expression levels may be performed using an oligonucleotide array, e.g., be isolating the one or more biomarkers that are nucleic acids from the sample and hybridizing them with known nucleic acids on a solid support. The step of determining expression levels may also be performed using cDNA which is made using mRNA collected from the human cells as a template. In some embodiments, a detectable label may be used to label the biomarker and/or the target for biomarker binding (e.g., an antibody) that is used to determine expression levels. The step of screening may be accomplished by quantitating the mRNA, protein or both mRNA and protein level of the biomarker. Often, the biomarker may be detected at the mRNA level and may be quantitated by a method selected from the group consisting of polymerase chain reaction, real time polymerase chain reaction, reverse transcriptase polymerase chain reaction, hybridization, probe hybridization, and gene expression array. It may also be useful to screen by detection of a polymorphism in the biomarker. Other ways for determining the level of expression may be accomplished using at least one technique selected from the group consisting of polymerase chain reaction, heteroduplex analysis, single stand conformational polymorphism analysis, ligase chain reaction, comparative genome hybridization, Southern blotting, Northern blotting, Western blotting, enzyme-linked immunosorbent assay, fluorescent resonance energy-transfer and sequencing. The sample will often be blood, however, any of a number of cells may be used as well, e.g., leukocytes, biopsy cells, cells in fluids or secretions and the like. In some embodiments, the biomarker may be proteins extracted from blood.

Yet another embodiment of the present invention includes a method of identifying a human subject suspected of having an infectious disease by determining the expression level of a biomarker having one or more of the following genes for the listed target: genes overexpressed as a result of a bacterial versus a viral infection: Translational elongation; EEF1G; Regulation of translational initiation; EIF3S5; EIF3S7; EIF4B; Protein biosynthesis; QARS; RPL31; RPL4; Regulation of transcription; PFDN5; Cell adhesion; CD44; Metabolism; HADHA; PCBP2; Miscellaneous; dJ507115.1. The step of determining expression levels is performed by measuring amounts of mRNA expressed by the set of genes or even by measuring amounts of protein expressed by the set of genes.

Yet another method of identifying a human subject suspected of having an infectious disease wherein overexpression of the following genes is indicative of S. aureus infection: Signal Transduction; CXCL1; JAG1; RGS2; Metabolism; GAPD; PPIB; PSMA7; MMP9; p44S10; Protein Targeting; TRAM2; Intracellular Protein Transport; SEC24C; Miscellaneous; ACTG1; CGI-96; MGC2963; STAU.

Yet another method of identifying a human subject suspected of having an infectious disease wherein overexpression of the following genes is indicative of E. coli infection: Intracellular signaling; RASA 1; SNX4; Regulation of translational initiation; AF1Q; Regulation of transcription; SMAD2; Cell adhesion; JUP; Metabolism; PP; MAN1C1; Miscellaneous; FLJ10287; FLJ20152; LRRN3; LRRN3; SGPP1; UBAP2L.

Yet another method of the present invention includes a computer implemented method for determining the genotype of a sample by, obtaining a plurality of sample probe intensities; diagnosing an infectious disease based upon the sample probe intensities; calculating linear correlation coefficient between the sample probe intensities and reference probe intensities; and accepting the tentative genotype as the genotype of the sample if the linear correlation coefficient is greater than a threshold value. In certain embodiment the threshold value may be between about 0.7 to about 1 or more, however, certain threshold values includes is at least 0.8; at least 0.9 and/or at least 0.95. The probe intensities may be selected from a gene expression profile from the tissue sample wherein expression of the two or more of the following genes is measured for the listed target:

S. aureus: Signal Transduction; CXCL1; JAGI; RGS2; Metabolism; GAPD; PPIB; PSMA7; MMP9; p44S10; Protein Targeting; TRAM2; Intracellular Protein Transport; SEC24C; Miscellaneous; ACTG1; CGI-96; MGC2963; STAU; and combinations thereof;

E. coli: Intracellular signaling; RASA1; SNX4; Regulation of translational initiation; AF1Q; Regulation of transcription; SMAD2; Cell adhesion; JUP; Metabolism; PP; MAN1C1; Miscellaneous; FLJ10287; FLJ20152; LRRN3; LRRN3; SGPP1; UBAP2L; and combinations thereof; and

Influenza: Response to virus; cig5; DNAPTP6; IF127; IF135; IF144; IF144; OAS1; Immune response; BST2; G1P2; LY6E; MX1; Anti-apoptosis; SON; Cell growth and/or maintenance; TRIM14; Miscellaneous; APOBEC3C; Clorf29; FLJ20035; FLJ38348; HSXIAPAF1; KIAA0152; PHACTR2; USP18; ZBP1; and combinations thereof.

Another embodiment of the present invention is a computer readable medium that includes computer-executable instructions for performing the method for determining the genotype of a sample comprising: obtaining a plurality of sample probe intensities; diagnosing an infectious disease based upon the sample probe intensities for six or more genes selected those genes listed in Tables 1, 4, 5 and/or Supplemental Tables 1 to 11 and combinations thereof; and calculating a linear correlation coefficient between the sample probe intensities and reference probe intensities; and accepting the tentative genotype as the genotype of the sample if the linear correlation coefficient is greater than a threshold value.

Another embodiment of the present invention is a system for identifying a host immune response to an infectious disease that includes a microarray for the detection of gene expression, wherein the microarray comprises four or more biomarker selected from selected those genes listed in Table 4, Table 5, and Supplemental Tables 1 to 11 and combinations thereof, wherein the gene expression data obtained from the microarray correlates to the host immune response to an infectious disease with a threshold value.

Another embodiment of the present invention is a system for diagnosing an infectious disease by obtaining gene expression data from a microarray; and determining the expression four or more biomarkers selected from the group consisting of four or more genes selected from Tables 1, 4 and/or 5, wherein the gene expression data obtained from the microarray correlates to a host immune response to the infectious disease with a threshold value of at least 0.8. For use with the system of the present invention, the biomarkers may be selected from 5, 6, 7, 8, 9, 10, 11, 12 or 13 genes or gene modules and from one or more of the Supplementary Tables, and combinations thereof, incorporated herein by reference.

Another embodiment is a prognostic gene array that is a customized gene array that includes a combination of genes that are representative of one or more transcriptional modules, wherein the transcriptome of a patient that is contacted with the customized gene array is prognostic of SLE. The array may be used to monitor the patient's response to therapy for SLE. The array may also be used to distinguish between an autoimmune disease, a viral infection a bacterial infection, cancer and transplant rejection. For certain direct measurement purposes the array may even be organized into two or more transcriptional modules that may be visually scanned and the extent of expression analyzed optically, e.g., with the naked eye and/or with image processing equipment. For example, the array may be organized into three transcriptional modules with one or more submodules selected from 5, 6, 7, 8, 9, 10, 11, 12 or 13 genes or gene modules and from one or more of the Supplementary Tables, and combinations thereof, wherein probes that bind specifically to one or more of the genes are selected from within the three or more modules and are indicative of an infectious disease or other condition, as disclosed herein.

Another embodiment of the present invention includes a method for selecting patients for a clinical trial by obtaining the transcriptome of a prospective patient; comparing the transcriptome to one or more transcriptional modules that are indicative of a disease or condition that is to be treated in the clinical trial; and determining the likelihood that a patient is a good candidate for the clinical trial based on the presence, absence or level of one or more genes that are expressed in the patient's transcriptome within one or more transcriptional modules that are correlated with success in a clinical trial. For use with the method, each module may include a vector that correlates with a sum of the proportion of transcripts in a sample; a vector wherein one or more diseases or conditions are associated with the one or more vectors; a vector that correlates to the expression level of one or more genes within each module and/or a vector that includes modules for the detection, characterization, diagnosis, prognosis and/or monitoring of normal versus patients infected with an infectious disease or a congenital, degenerative, acquired or other disease.

### BRIEF DESCRIPTION OF THE DRAWINGS

For a more complete understanding of the features and advantages of the present invention, reference is now made to the detailed description of the invention along with the accompanying figures and in which:

FIG. 1 shows that it is possible to differentiate between patients with influenza A virus infection from patients with bacterial infections. FIG. 1a shows the hierarchical clustering of 854 genes obtained from Mann-Whitney rank test comparison (p<0.01) between two groups: influenza A (Inf A, 11 samples, green rectangle) and bacterial infections (red rectangle) with Escherichia coli (E. coli, 6 samples) or Streptococcus pneumoniae (S.pn, 6 samples). Transformed expression levels are indicated by color scale, with red representing relatively high expression and blue indicating relatively low expression compared to the median expression for each gene across all donors. The black bar indicates interferon-inducible genes (IFN), and the blue bar indicates genes involved in protein biosynthesis. Genes are listed in Supplementary Table 2. FIG. 1b shows the results from a supervised learning algorithm was used to identify 35 genes presenting the highest capacity to discriminate the two classes (Table 1 and Supplementary Table 3). Leave-one-out cross-validation of the training set with 35 genes classified the samples with 91% accuracy. The predicted class is indicated by light colored solid rectangles (green for influenza A and red for bacteria). Two patients with bacterial infections were misclassified. FIG. 1c shows a summary of the 35 classifier genes thus identified were tested on an independent set of patients (open rectangles), including 7 new patients with influenza A (green), 23 with E. coli (red) and 7 with S. pneumoniae infections. The 37 samples in this test set were classified with 95% accuracy (predicted class is indicated by light colored rectangles). One patient was misclassified and one patient was indeterminate in class prediction (gray box). FIG. 1d shows the 35 classifier genes identified in 7b that were tested on an independent set of patients (open squares), including 7 new patients with influenza A (Inf A), and 31 with S. aureus infections. The 38 samples were classified with 87% accuracy.

FIG. 2 shows the expression levels of the 35 classifier genes discriminating patients with Influenza A infection from patients with bacterial infections. Scaled gene expression values (Average Difference intensity) are plotted for the 35 classifier genes represented in FIG. 7b that discriminate between samples from patients with influenza A (11 samples, green squares) and bacterial infections (6 samples with E. coli and 6 samples with S. pneumoniae, red diamonds). Each plot represents one sample, lines represent median expression.

FIG. 3a to 3e shows that it is possible to differentiate between patients with S. aureus infections from patients with E. coli infections. FIG. 9a shows the hierarchical clustering of 211 genes obtained from Mann-Whitney rank test comparison (p<0.01) between two groups: Staphylococcus aureus (S. aureus, 10 samples, red rectangle) and Escherichia coli (E. coli 10 samples, blue rectangle) infections. Transformed expression levels are indicated by color scale, with red representing relative high expression and blue indicating relative low expression compared to the median expression for each gene across all donors. Genes are listed in Supplementary Table 4. FIG. 3b shows the results from a supervised learning algorithm was used to identify 30 genes presenting the highest capacity to discriminate the two classes (see also Supplementary Table 6). Leave-one-out cross-validation of the training set with 30 classifier genes grouped the samples with 95% accuracy. FIG. 3c shows that the 30 classifier genes thus identified were tested on an independent set of patients (open rectangles), including 21 new patients with S. aureus and 19 with E. coli infections. The 40 samples in this test set were predicted with 85% accuracy (predicted class is indicated by light colored rectangles). Of these 40 samples, only 2 were misclassified, while the class of four other samples could not be determined (open rectangles).

FIGS. 3d and 3e show the validation of differentially expressed genes by real-time RT-PCR. FIG. 3d shows the levels of expression of 9 genes were measured by real-time RT-PCR in samples obtained from patients with S. aureus (Sa) or E. coli (Ec) infections (fold change in gene expression over healthy controls, log transformed except for RGS2, FCAR and ALOX). Each plot represents one sample, lines represent median expression. FIG. 9e shows the correlation between expression values obtained by real-time RT-PCR analysis (abscissa) and microarray analysis (ordinate—normalized to the expression in the sample from the same healthy control to which real-time RT-PCR data were normalized; log scale). See Supplementary Table 5 for details.

FIG. 4a to 4e show the expression levels of the 30 classifier genes discriminating patients with E. coli infections from patients with S. aureus infections. Scaled gene expression values (Average Difference intensity) are plotted for the 30 classifier genes represented in FIG. 3b that discriminate between samples from patients with E. coli (10 samples, blue squares) and S. aureus infections (10 samples, red diamonds). Each plot represents one sample, lines represent median expression. FIGS. 4b to 4e show that the present invention may be used to discern between patients with bacterial infections. FIG. 4b shows hierarchical clustering of 242 genes obtained from Mann-Whitney rank test comparison (p<0.01) between groups of patients with E. coli infections (11 samples) or S. pneumoniae infections (11 samples). Transformed expression levels are indicated by color scale, with red representing relative high expression and blue indicating relative low expression compared to the median expression for each gene across all donors. Genes are listed in Supplementary Table 7. FIG. 4c shows the results from a supervised learning algorithm was used to identify genes representing the highest capacity to discriminate the two classes. Leave-one-out cross-validation of the training set with 45 predictor genes classified the samples with 85% (20/22) accuracy. Classifier genes are listed in Supplementary Table 8. FIG. 4d shows the results from an unsupervised hierarchical clustering of 127 genes obtained from Mann-Whitney rank test comparison (p<0.01) between groups of patients with S. aureus infection (12 samples) or S. pneumoniae infection (11 samples). Transformed expression levels are indicated by color scale, with red representing relative high expression and blue indicating relative low expression compared to the median expression for each gene across all donors. Genes are listed in Supplementary Table 9 FIG. 4e shows a supervised learning algorithm was used to identify genes presenting the highest capacity to discriminate the two classes. Leave-one-out cross-validation of the training set with 30 genes classified the samples with 83% (19/23) accuracy. Classifier genes are listed in Supplementary Table 10.

FIG. 5 shows the distinctive patterns of gene expression in circulating leukocytes obtained from patients with acute respiratory infections. FIG. 5a shows uses the 30 classifier genes found to discriminate S. aureus from E. coli (Venn diagram, right: Sa from Ec; FIG. 2 and Supplementary Table 6), to identify 30 genes that distinguish S. aureus from S. pneumoniae (Venn diagram, left: Sa from Sp; FIG. 5a and Supplementary Table 10) and 45 genes that distinguish E. coli from S. pneumoniae (Venn diagram, bottom: Ec from Sp; Supplementary FIG. 5b and Supplementary Table 8). Only 3 genes were shared between either of these groups. In FIG. 5b the three groups of genes found to discriminate samples from patients with bacterial infections shown in FIG. 5a were merged (102 unique genes, Venn diagram, left) and compared to the classifier genes used to discriminate influenza A from bacterial infections (35 genes, Venn diagram, right; FIG. 5b and Supplementary Table 3). No genes were shared between these two groups. FIG. 5c shows the 137 classifier genes that discriminate Influenza A from bacterial infections and the three groups of patients with different bacterial infections were merged and used to generate discriminatory patterns of expression among 27 patients with respiratory infections and 7 healthy volunteers. Values were normalized to the median expression of each gene across all donors. Clustering of conditions partitioned samples into four major groups. Four samples belonging to the influenza A group and one from the S. aureus formed a distinct subgroup characterized by a mixed signature (*).

FIG. 6 shows an analysis of significance patterns for infectious disease monitoring. Gene expression levels measured in each group of patients were compared to results obtained in control groups formed by healthy volunteers (Mann Whitney U test). Selection criteria were then applied to p-values generated for patients with Influenza A (FLU) or Systemic Lupus Erythematosus (SLE). Left column: over-expressed genes; Right column: under-expressed genes; Upper row: significantly changed in both FLU and SLE (p<0.01); Middle row: significantly changed in SLE (p<0.01), not FLU (p>0.5); Bottom row: significantly changed in FLU (p<0.01), not SLE (p>0.5). Genes were arranged by hierarchical clustering of p-values. Color scale: Green indicates low p-values, yellow and white high p-values. Blue branches of the dendrograms indicate disease-specific signatures (C1-C4; see Supplementary Table 11 for details).

FIG. 7 shows gene vectors that may be used for mapping transcriptional changes at the module-levels identifies disease-specific patterns.

FIG. 8 shows the microarray scores for the assessment of disease severity in patients with acute infections.

FIGS. 9a to 9c summarize independent confirmation and validation across microarray platforms.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. Unless defined otherwise, all technical and scientific terms used herein have the meaning commonly understood by a person skilled in the art to which this invention belongs. The following references provide one of skill with a general definition of many of the terms used in this invention: Singleton, et al., Dictionary Of Microbiology And Molecular Biology (2d ed. 1994); The Cambridge Dictionary Of Science And Technology (Walker ed., 1988); The Glossary Of Genetics, 5th Ed., R. Rieger et al. (eds.), Springer Verlag (1991); and Hale & Marham, The Harper Collins Dictionary Of Biology (1991).

Various biochemical and molecular biology methods are well known in the art. For example, methods of isolation and purification of nucleic acids are described in detail in WO 97/10365, WO 97/27317, Chapter 3 of Laboratory Techniques in Biochemistry and Molecular Biology: Hybridization With Nucleic Acid Probes, Part I. Theory and Nucleic Acid Preparation, (P. Tijssen, ed.) Elsevier, N.Y. (1993); Chapter 3 of Laboratory Techniques in Biochemistry and Molecular Biology Hybridization With Nucleic Acid Probes, Part 1. Theory and Nucleic Acid Preparation, (P. Tijssen, ed.) Elsevier, N.Y. (1993); and Sambrook et al., Molecular Cloning: A Laboratory Manual, Cold Spring Harbor Press, N.Y., (1989); and Current Protocols in Molecular Biology, (Ausubel, F. M. et al., eds.) John Wiley & Sons, Inc., New York (1987-1999), including supplements such as supplement 46 (April 1999).

Bioinformatics Definitions

As used herein, an “object” refers to any item or information of interest (generally textual, including noun, verb, adjective, adverb, phrase, sentence, symbol, numeric characters, etc.). Therefore, an object is anything that can form a relationship and anything that can be obtained, identified, and/or searched from a source. “Objects” include, but are not limited to, an entity of interest such as gene, protein, disease, phenotype, mechanism, drug, etc. In some aspects, an object may be data, as further described below.

As used herein, a “relationship” refers to the co-occurrence of objects within the same unit (e.g., a phrase, sentence, two or more lines of text, a paragraph, a section of a webpage, a page, a magazine, paper, book, etc.). It may be text, symbols, numbers and combinations, thereof.

As used herein, “meta data content” refers to information as to the organization of text in a data source. Meta data can comprise standard metadata such as Dublin Core metadata or can be collection-specific. Examples of metadata formats include, but are not limited to, Machine Readable Catalog (MARC) records used for library catalogs, Resource Description Format (RDF) and the Extensible Markup Language (XML). Meta objects may be generated manually or through automated information extraction algorithms.

As used herein, an “engine” refers to a program that performs a core or essential function for other programs. For example, an engine may be a central program in an operating system or application program that coordinates the overall operation of other programs. The term “engine” may also refer to a program containing an algorithm that can be changed. For example, a knowledge discovery engine may be designed so that its approach to identifying relationships can be changed to reflect new rules of identifying and ranking relationships.

As used herein, “statistical analysis” refers to a technique based on counting the number of occurrences of each term (word, word root, word stem, n-gram, phrase, etc.). In collections unrestricted as to subject, the same phrase used in different contexts may represent different concepts. Statistical analysis of phrase co-occurrence can help to resolve word sense ambiguity. “Syntactic analysis” can be used to further decrease ambiguity by part-of-speech analysis. As used herein, one or more of such analyses are referred to more generally as “lexical analysis.” “Artificial intelligence (AI)” refers to methods by which a non-human device, such as a computer, performs tasks that humans would deem noteworthy or “intelligent.” Examples include identifying pictures, understanding spoken words or written text, and solving problems.

As used herein, the term “database” refers to repositories for raw or compiled data, even if various informational facets can be found within the data fields. A database is typically organized so its contents can be accessed, managed, and updated (e.g., the database is dynamic). The term “database” and “source” are also used interchangeably in the present invention, because primary sources of data and information are databases. However, a “source database” or “source data” refers in general to data, e.g., unstructured text and/or structured data, that are input into the system for identifying objects and determining relationships. A source database may or may not be a relational database. However, a system database usually includes a relational database or some equivalent type of database which stores values relating to relationships between objects.

As used herein, a “system database” and “relational database” are used interchangeably and refer to one or more collections of data organized as a set of tables containing data fitted into predefined categories. For example, a database table may comprise one or more categories defined by columns (e.g. attributes), while rows of the database may contain a unique object for the categories defined by the columns. Thus, an object such as the identity of a gene might have columns for its presence, absence and/or level of expression of the gene. A row of a relational database may also be referred to as a “set” and is generally defined by the values of its columns. A “domain” in the context of a relational database is a range of valid values a field such as a column may include.

As used herein, a “domain of knowledge” refers to an area of study over which the system is operative, for example, all biomedical data. It should be pointed out that there is advantage to combining data from several domains, for example, biomedical data and engineering data, for this diverse data can sometimes link things that cannot be put together for a normal person that is only familiar with one area or research/study (one domain). A “distributed database” refers to a database that may be dispersed or replicated among different points in a network.

Terms such “data” and “information” are often used interchangeably, as are “information” and “knowledge.” As used herein, “data” is the most fundamental unit that is an empirical measurement or set of measurements. Data is compiled to contribute to information, but it is fundamentally independent of it. Information, by contrast, is derived from interests, e.g., data (the unit) may be gathered on ethnicity, gender, height, weight and diet for the purpose of finding variables correlated with risk of cardiovascular disease. However, the same data could be used to develop a formula or to create “information” about dietary preferences, i.e., likelihood that certain products in a supermarket have a higher likelihood of selling.

As used herein, “information” refers to a data set that may include numbers, letters, sets of numbers, sets of letters, or conclusions resulting or derived from a set of data. “Data” is then a measurement or statistic and the fundamental unit of information. “Information” may also include other types of data such as words, symbols, text, such as unstructured free text, code, etc. “Knowledge” is loosely defined as a set of information that gives sufficient understanding of a system to model cause and effect. To extend the previous example, information on demographics, gender and prior purchases may be used to develop a regional marketing strategy for food sales while information on nationality could be used by buyers as a guideline for importation of products. It is important to note that there are no strict boundaries between data, information, and knowledge; the three terms are, at times, considered to be equivalent. In general, data comes from examining, information comes from correlating, and knowledge comes from modeling.

As used herein, “a program” or “computer program” refers generally to a syntactic unit that conforms to the rules of a particular programming language and that is composed of declarations and statements or instructions, divisible into, “code segments” needed to solve or execute a certain function, task, or problem. A programming language is generally an artificial language for expressing programs.

As used herein, a “system” or a “computer system” generally refers to one or more computers, peripheral equipment, and software that perform data processing. A “user” or “system operator” in general includes a person, that uses a computer network accessed through a “user device” (e.g., a computer, a wireless device, etc) for the purpose of data processing and information exchange. A “computer” is generally a functional unit that can perform substantial computations, including numerous arithmetic operations and logic operations without human intervention.

As used herein, “application software” or an “application program” refers generally to software or a program that is specific to the solution of an application problem. An “application problem” is generally a problem submitted by an end user and requiring information processing for its solution.

As used herein, a “natural language” refers to a language whose rules are based on current usage without being specifically prescribed, e.g., English, Spanish or Chinese. As used herein, an “artificial language” refers to a language whose rules are explicitly established prior to its use, e.g., computer-programming languages such as C, C++, Java, BASIC, FORTRAN, or COBOL.

As used herein, “statistical relevance” refers to using one or more of the ranking schemes (O/E ratio, strength, etc.), where a relationship is determined to be statistically relevant if it occurs significantly more frequently than would be expected by random chance.

As used herein, the terms “coordinately regulated genes” or “transcriptional modules” are used interchangeably to refer to grouped, gene expression profiles (e.g., signal values associated with a specific gene sequence) of specific genes. A value may be assigned to the combination of one or more “coordinately regulated genes” to provide a “transcriptome value vector” or “transcriptome vector” that may be expressed as a single value. For example, the value may be provided numerically, plotted in a spider chart, plotted with various intensities, color(s), values or as a contours, e.g., an elevation plot. Each transcriptional module may correlate with one or more pieces of data, e.g., a literature search portion and actual empirical gene expression value data obtained from a gene microarray. The set of genes that is selected into a transcriptional modules is based on the analysis of gene expression data (module extraction algorithm described above). Additional steps are taught by Chaussabel, D. & Sher, A. Mining microarray expression data by literature profiling. Genome Biol 3, RESEARCH0055 (2002), (http://genomebiology.com/2002/3/10/research/0055) relevant portions incorporated herein by reference and expression data obtained from a disease or condition of interest, e.g., Systemic Lupus erythematosus, arthritis, lymphoma, carcinoma, melanoma, acute infection, autoimmune disorders, autoinflammatory disorders, etc.).

The Table below lists examples of keywords that were used to develop the literature search portion or contribution to the transcription modules. The skilled artisan will recognize that other terms may easily be selected for other conditions, e.g., specific cancers, specific infectious disease, transplantation, etc. For example, genes and signals for those genes associated with T cell activation are described hereinbelow as Module ID “M 2.8” in which certain keywords (e.g., Lymphoma, T-cell, CD4, CD8, TCR, Thymus, Lymphoid, IL2) were used to identify key T-cell associated genes, e.g., T-cell surface markers (CD5, CD6, CD7, CD26, CD28, CD96); molecules expressed by lymphoid lineage cells (lymphotoxin beta, IL2-inducible T-cell kinase, TCF7; and T-cell differentiation protein mal, GATA3, STAT5B). Next, the complete module is developed by correlating data from a patient population for these genes (regardless of platform, presence/absence and/or up or downregulation) to generate the transcriptional module. In some cases, the gene profile does not match (at this time) any particular clustering of genes for these disease conditions and data, however, certain physiological pathways (e.g., cAMP signaling, zinc-finger proteins, cell surface markers, etc.) are found within the “Underdetermined” modules. In fact, the gene expression data set may be used to extract genes that have coordinated expression prior to matching to the keyword search, i.e., either data set may be correlated prior to cross-referencing with the second data set.

TABLE 1
Examples of Genes within Distinct Modules

| Module | Number of | | |
|---|---|---|---|
| I.D. | probe sets | Keyword selection | Assessment |
| M 1.1 | 76 | Ig, Immunoglobulin, | Plasma cells. Includes genes coding for Immunoglobulin |
| | | Bone, Marrow, PreB, | chains (e.g. IGHM, IGJ, IGLL1, IGKC, IGHD) and the |
| | | IgM, Mu. | plasma cell marker CD38. |
| M 1.2 | 130 | Platelet, Adhesion, | Platelets. Includes genes coding for platelet glycoproteins |
| | | Aggregation, | (ITGA2B, ITGB3, GP6, GP1A/B), and platelet-derived |
| | | Endothelial, Vascular | immune mediators such as PPPB (pro-platelet basic protein) |
| | | | and PF4 (platelet factor 4). |
| M 1.3 | 80 | Immunoreceptor, | B-cells. Includes genes coding for B-cell surface markers |
| | | BCR, B-cell, IgG | (CD72, CD79A/B, CD19, CD22) and other B-cell associated |
| | | | molecules: Early B-cell factor (EBF), B-cell linker (BLNK) |
| | | | and B lymphoid tyrosine kinase (BLK). |
| M 1.4 | 132 | Replication, | Undetermined. This set includes regulators and targets of |
| | | Repression, Repair, | cAMP signaling pathway (JUND, ATF4, CREM, PDE4, |
| | | CREB, Lymphoid, | NR4A2, VIL2), as well as repressors of TNF-alpha mediated |
| | | TNF-alpha | NF-KB activation (CYLD, ASK, TNFAIP3). |
| M 1.5 | 142 | Monocytes, | Myeloid lineage. Includes molecules expressed by cells of |
| | | Dendritic, MHC, | the myeloid lineage (CD86, CD163, FCGR2A), some of |
| | | Costimulatory, | which being involved in pathogen recognition (CD14, TLR2, |
| | | TLR4, MYD88 | MYD88). This set also includes TNF family members |
| | | | (TNFR2, BAFF). |
| M 1.6 | 141 | Zinc, Finger, P53, | Undetermined. This set includes genes coding for signaling |
| | | RAS | molecules, e.g. the zinc finger containing inhibitor of |
| | | | activated STAT (PIAS1 and PIAS2), or the nuclear factor of |
| | | | activated T-cells NFATC3. |
| M 1.7 | 129 | Ribosome, | MHC/Ribosomal proteins. Almost exclusively formed by |
| | | Translational, 40S, | genes coding MHC class I molecules (HLA-A,B,C,G,E) + Beta |
| | | 60S, HLA | 2-microglobulin (B2M) or Ribosomal proteins (RPLs, |
| | | | RPSs). |
| M 1.8 | 154 | Metabolism, | Undetermined. Includes genes encoding metabolic enzymes |
| | | Biosynthesis, | (GLS, NSF1, NAT1) and factors involved in DNA |
| | | Replication, Helicase | replication (PURA, TERF2, EIF2S1). |
| M 2.1 | 95 | NK, Killer, Cytolytic, | Cytotoxic cells. Includes cytotoxic T-cells amd NK-cells |
| | | CD8, Cell-mediated, | surface markers (CD8A, CD2, CD160, NKG7, KLRs), |
| | | T-cell, CTL, IFN-g | cytolytic molecules (granzyme, perforin, granulysin), |
| | | | chemokines (CCL5, XCL1) and CTL/NK-cell associated |
| | | | molecules (CTSW). |
| M 2.2 | 49 | Granulocytes, | Neutrophils. This set includes innate molecules that are found |
| | | Neutrophils, Defense, | in neutrophil granules (Lactotransferrin: LTF, defensin: |
| | | Myeloid, Marrow | DEAF1, Bacterial Permeability Increasing protein: BPI, |
| | | | Cathelicidin antimicrobial protein: CAMP . . . ). |
| M 2.3 | 148 | Erythrocytes, Red, | Erythrocytes. Includes hemoglobin genes (HGBs) and other |
| | | Anemia, Globin, | erythrocyte-associated genes (erythrocytic alkirin: ANK1, |
| | | Hemoglobin | Glycophorin C: GYPC, hydroxymethylbilane synthase: |
| | | | HMBS, erythroid associated factor: ERAF). |
| M 2.4 | 133 | Ribonucleoprotein, | Ribosomal proteins. Including genes encoding ribosomal |
| | | 60S, nucleolus, | proteins (RPLs, RPSs), Eukaryotic Translation Elongation |
| | | Assembly, | factor family members (EEFs) and Nucleolar proteins |
| | | Elongation | (NPM1, NOAL2, NAP1L1). |
| M 2.5 | 315 | Adenoma, Interstitial, | Undetermined. This module includes genes encoding |
| | | Mesenchyme, | immune-related (CD40, CD80, CXCL12, IFNA5, IL4R) as |
| | | Dendrite, Motor | well as cytoskeleton-related molecules (Myosin, Dedicator of |
| | | | Cytokenesis, Syndecan 2, Plexin C1, Distrobrevin). |
| M 2.6 | 165 | Granulocytes, | Myeloid lineage. Includes genes expressed in myeloid |
| | | Monocytes, Myeloid, | lineage cells (IGTB2/CD18, Lymphotoxin beta receptor, |
| | | ERK, Necrosis | Myeloid related proteins 8/14 Formyl peptide receptor 1), |
| | | | such as Monocytes and Neutrophils. |
| M 2.7 | 71 | No keywords | Undetermined. This module is largely composed of |
| | | extracted. | transcripts with no known function. Only 20 genes associated |
| | | | with literature, including a member of the chemokine-like |
| | | | factor superfamily (CKLFSF8). |
| M 2.8 | 141 | Lymphoma, T-cell, | T-cells. Includes T-cell surface markers (CD5, CD6, CD7, |
| | | CD4, CD8, TCR, | CD26, CD28, CD96) and molecules expressed by lymphoid |
| | | Thymus, Lymphoid, | lineage cells (lymphotoxin beta, IL2-inducible T-cell kinase, |
| | | IL2 | TCF7, T-cell differentiation protein mal, GATA3, |
| | | | STAT5B). |
| M 2.9 | 159 | ERK, | Undetermined. Includes genes encoding molecules that |
| | | Transactivation, | associate to the cytoskeleton (Actin related protein 2/3, |
| | | Cytoskeletal, MAPK, | MAPK1, MAP3K1, RAB5A). Also present are T-cell |
| | | JNK | expressed genes (FAS, ITGA4/CD49D, ZNF1A1). |
| M 2.10 | 106 | Myeloid, | Undetermined. Includes genes encoding for Immune-related |
| | | Macrophage, | cell surface molecules (CD36, CD86, LILRB), cytokines |
| | | Dendritic, | (IL15) and molecules involved in signaling pathways (FYB, |
| | | Inflammatory, | TICAM2-Toll-like receptor pathway). |
| | | Interleukin | |
| M 2.11 | 176 | Replication, Repress, | Undetermined. Includes kinases (UHMK1, CSNK1G1, |
| | | RAS, | CDK6, WNK1, TAOK1, CALM2, PRKCI, ITPKB, SRPK2, |
| | | Autophosphorylation, | STK17B, DYRK2, PIK3R1, STK4, CLK4, PKN2) and RAS |
| | | Oncogenic | family members (G3BP, RAB14, RASA2, RAP2A, KRAS). |
| M 3.1 | 122 | ISRE, Influenza, | Interferon-inducible. This set includes interferon-inducible |
| | | Antiviral, IFN- | genes: antiviral molecules (OAS1/2/3L, GBP1, G1P2, |
| | | gamma, IFN-alpha, | EIF2AK2/PKR, MX1, PML), chemokines (CXCL10/IP-10), |
| | | Interferon | signaling molecules (STAT1, STAt2, IRF7, ISGF3G). |
| M 3.2 | 322 | TGF-beta, TNF, | Inflammation I. Includes genes encoding molecules involved |
| | | Inflammatory, | in inflammatory processes (e.g. IL8, ICAM1, C5R1, CD44, |
| | | Apoptotic, | PLAUR, IL1A, CXCL16), and regulators of apoptosis |
| | | Lipopolysaccharide | (MCL1, FOXO3A, RARA, BCL3/6/2A1, GADD45B). |
| M 3.3 | 276 | Inflammatory, | Inflammation II. Includes molecules inducing or inducible by |
| | | Defense, Lysosomal, | inflammation (IL18, ALOX5, ANPEP, AOAH, HMOX1, |
| | | Oxidative, LPS | SERPINB1), as well as lysosomal enzymes (PPT1, CTSB/S, |
| | | | NEU1, ASAH1, LAMP2, CAST). |
| M 3.4 | 325 | Ligase, Kinase, KIP1, | Undetermined. Includes protein phosphatases (PPP1R12A, |
| | | Ubiquitin, Chaperone | PTPRC, PPP1CB, PPM1B) and phosphoinositide 3-kinase |
| | | | (PI3K) family members (PIK3CA, PIK32A, PIP5K3). |
| M 3.5 | 22 | No keyword | Undetermined. Composed of only a small number of |
| | | extracted | transcripts. Includes hemoglobin genes (HBA1, HBA2, |
| | | | HBB). |
| M 3.6 | 288 | Ribosomal, T-cell, | Undetermined. This set includes mitochondrial ribosomal |
| | | Beta-catenin | proteins (MRPLs, MRPs), mitochondrial elongations factors |
| | | | (GFM1/2), Sortin Nexins (SN1/6/14) as well as lysosomal |
| | | | ATPases (ATP6V1C/D). |
| M 3.7 | 301 | Spliceosome, | Undetermined. Includes genes encoding proteasome subunits |
| | | Methylation, | (PSMA2/5, PSMB5/8); ubiquitin protein ligases HIP2, |
| | | Ubiquitin | STUB1, as well as components of ubiqutin ligase complexes |
| | | | (SUGT1). |
| M 3.8 | 284 | CDC, TCR, CREB, | Undetermined. Includes genes encoding enzymes: |
| | | Glycosylase | aminomethyltransferase, arginyltransferase, asparagines |
| | | | synthetase, diacylglycerol kinase, inositol phosphatases, |
| | | | methyltransferases, helicases . . . |
| M 3.9 | 260 | Chromatin, | Undetermined. Includes genes encoding kinases (IBTK, |
| | | Checkpoint, | PRKRIR, PRKDC, PRKCI) and phosphatases (e.g. PTPLB, |
| | | Replication, | PPP2CB/3CB, PTPRC, MTM1, MTMR2). |
| | | Transactivation | |

Biological Definitions

As used herein, the term “array” refers to a solid support or substrate with one or more peptides or nucleic acid probes attached to the support. Arrays typically have one or more different nucleic acid or peptide probes that are coupled to a surface of a substrate in different, known locations. These arrays, also described as “microarrays”, “gene-chips” or DNA chips that may have 10,000; 20,000, 30,000; or 40,000 different identifiable genes based on the known genome, e.g., the human genome. These pan-arrays are used to detect the entire “transcriptome” or transcriptional pool of genes that are expressed or found in a sample, e.g., nucleic acids that are expressed as RNA, mRNA and the like that may be subjected to RT and/or RT-PCR to made a complementary set of DNA replicons. Arrays may be produced using mechanical synthesis methods, light directed synthesis methods and the like that incorporate a combination of non-lithographic and/or photolithographic methods and solid phase synthesis methods. Bead arrays that include 50-mer oligonucleotide probes attached to 3 micrometer beads may be used that are, e.g., lodged into microwells at the surface of a glass slide or are part of a liquid phase suspension arrays (e.g., Luminex or Illumina) that are digital beadarrays in liquid phase and uses “barcoded” glass rods for detection and identification.

Various techniques for the synthesis of these nucleic acid arrays have been described, e.g., fabricated on a surface of virtually any shape or even a multiplicity of surfaces. Arrays may be peptides or nucleic acids on beads, gels, polymeric surfaces, fibers such as fiber optics, glass or any other appropriate substrate. Arrays may be packaged in such a manner as to allow for diagnostics or other manipulation of an all inclusive device, see for example, U.S. Pat. No. 6,955,788, relevant portions incorporated herein by reference.

As used herein, the term “disease” refers to a physiological state of an organism with any abnormal biological state of a cell. Disease includes, but is not limited to, an interruption, cessation or disorder of cells, tissues, body functions, systems or organs that may be inherent, inherited, caused by an infection, caused by abnormal cell function, abnormal cell division and the like. A disease that leads to a “disease state” is generally detrimental to the biological system, that is, the host of the disease. With respect to the present invention, any biological state, such as an infection (e.g., viral, bacterial, fungal, helminthic, etc.), inflammation, autoinflammation, autoimmunity, anaphylaxis, allergies, premalignancy, malignancy, surgical, transplantation, physiological, and the like that is associated with a disease or disorder is considered to be a disease state. A pathological state is generally the equivalent of a disease state.

Disease states may also be categorized into different levels of disease state. As used herein, the level of a disease or disease state is an arbitrary measure reflecting the progression of a disease or disease state as well as the physiological response upon, during and after treatment. Generally, a disease or disease state will progress through levels or stages, wherein the affects of the disease become increasingly severe. The level of a disease state may be impacted by the physiological state of cells in the sample.

As used herein, the terms “therapy” or “therapeutic regimen” refer to those medical steps taken to alleviate or alter a disease state, e.g., a course of treatment intended to reduce or eliminate the affects or symptoms of a disease using pharmacological, surgical, dietary and/or other techniques. A therapeutic regimen may include a prescribed dosage of one or more drugs or surgery. Therapies will most often be beneficial and reduce the disease state but in many instances the effect of a therapy will have non-desirable or side-effects. The effect of therapy will also be impacted by the physiological state of the host, e.g., age, gender, genetics, weight, other disease conditions, etc.

As used herein, the term “pharmacological state” or “pharmacological status” refers to those samples that will be, are and/or were treated with one or more drugs, surgery and the like that may affect the pharmacological state of one or more nucleic acids in a sample, e.g., newly transcribed, stabilized and/or destabilized as a result of the pharmacological intervention. The pharmacological state of a sample relates to changes in the biological status before, during and/or after drug treatment and may serve a diagnostic or prognostic function, as taught herein. Some changes following drug treatment or surgery may be relevant to the disease state and/or may be unrelated side-effects of the therapy. Changes in the pharmacological state are the likely results of the duration of therapy, types and doses of drugs prescribed, degree of compliance with a given course of therapy, and/or un-prescribed drugs ingested.

As used herein, the term “biological state” refers to the state of the transcriptome (that is the entire collection of RNA transcripts) of the cellular sample isolated and purified for the analysis of changes in expression. The biological state reflects the physiological state of the cells in the sample by measuring the abundance and/or activity of cellular constituents, characterizing according to morphological phenotype or a combination of the methods for the detection of transcripts.

As used herein, the term “expression profile” refers to the relative abundance of RNA, DNA or protein abundances or activity levels. The expression profile can be a measurement for example of the transcriptional state or the translational state by any number of methods and using any of a number of gene-chips, gene arrays, beads, multiplex PCR, quantitative PCR, run-on assays, Northern blot analysis, Western blot analysis, protein expression, fluorescence activated cell sorting (FACS), enzyme linked immunosorbent assays (ELISA), chemiluminescence studies, enzymatic assays, proliferation studies or any other method, apparatus and system for the determination and/or analysis of gene expression that are readily commercially available.

As used herein, the term “transcriptional state” of a sample includes the identities and relative abundances of the RNA species, especially mRNAs present in the sample. The entire transcriptional state of a sample, that is the combination of identity and abundance of RNA, is also referred to herein as the transcriptome. Generally, a substantial fraction of all the relative constituents of the entire set of RNA species in the sample are measured.

As used herein, the terms “transcriptional vectors,” “expression vectors,” and “genomic vectors” (used interchangeably) refers to transcriptional expression data that reflects the “proportion of differentially expressed genes.” For example, for each module the proportion of transcripts differentially expressed between at least two groups (e.g., healthy subjects vs patients). This vector is derived from the comparison of two groups of samples. The first analytical step is used for the selection of disease-specific sets of transcripts within each module. Next, there is the “expression level.” The group comparison for a given disease provides the list of differentially expressed transcripts for each module. It was found that different diseases yield different subsets of modular transcripts. With this expression level it is then possible to calculate vectors for each module(s) for a single sample by averaging expression values of disease-specific subsets of genes identified as being differentially expressed. This approach permits the generation of maps of modular expression vectors for a single sample, e.g., those described in the module maps disclosed herein. These vector module maps represent an averaged expression level for each module (instead of a proportion of differentially expressed genes) that can be derived for each sample. These composite “expression vectors” are formed through successive rounds of selection: 1) of the modules that were significantly changed across study groups and 2) of the genes within these modules which are significantly changed across study groups. Expression levels are subsequently derived by averaging the values obtained for the subset of transcripts forming each vector. Patient profiles can then be represented by plotting expression levels obtained for each of these vectors on a graph (e.g. on a radar plot). Therefore a set of vectors results from two round of selection, first at the module level, and then at the gene level. Vector expression values are composite by construction as they derive from the average expression values of the transcript forming the vector.

Using the present invention it is possible to identify and distinguish diseases not only at the module-level, but also at the gene-level; i.e., two diseases can have the same vector (identical proportion of differentially expressed transcripts, identical “polarity”), but the gene composition of the expression vector can still be disease-specific. This disease-specific customization permits the user to optimize the performance of a given set of markers by increasing its specificity.

Using modules as a foundation grounds expression vectors to coherent functional and transcriptional units containing minimized amounts of noise. Furthermore, the present invention takes advantage of composite transcriptional markers. As used herein, the term “composite transcriptional markers” refers to the average expression values of multiple genes (subsets of modules) as compared to using individual genes as markers (and the composition of these markers can be disease-specific). The composite transcriptional markers approach is unique because the user can develop multivariate microarray scores to assess disease severity in patients with, e.g., a viral, bacterial or other infectious disease, or to derive expression vectors disclosed herein. The fact that expression vectors are composite (i.e. formed by a combination of transcripts) further contributes to the stability of these markers. Most importantly, it has been found that using the composite modular transcriptional markers of the present invention the results found herein are reproducible across microarray platform, thereby providing greater reliability for regulatory approval. Indeed, vector expression values proved remarkably robust, as indicated by the excellent reproducibility obtained across microarray platforms; as well as the validation results obtained in an independent set of pediatric lupus patients. These results are of importance since improving the reliability of microarray data is a prerequisite for the widespread use of this technology in clinical practice (see, e.g., FDA MAQC program, which aims at establishing reproducibility across array platforms.).

Gene expression monitoring systems for use with the present invention may include customized gene arrays with a limited and/or basic number of genes that are specific and/or customized for the one or more target diseases. Unlike the general, pan-genome arrays that are in customary use, the present invention provides for not only the use of these general pan-arrays for retrospective gene and genome analysis without the need to use a specific platform, but more importantly, it provides for the development of customized arrays that provide an optimal gene set for analysis without the need for the thousands of other, non-relevant genes. One distinct advantage of the optimized arrays and modules of the present invention over the existing art is a reduction in the financial costs (e.g., cost per assay, materials, equipment, time, personnel, training, etc.), and more importantly, the environmental cost of manufacturing pan-arrays where the vast majority of the data is irrelevant. The modules of the present invention allow for the first time the design of simple, custom arrays that provide optimal data with the least number of probes while maximizing the signal to noise ratio. By eliminating the total number of genes for analysis, it is possible to, e.g., eliminate the need to manufacture thousands of expensive platinum masks for photolithography during the manufacture of pan-genetic chips that provide vast amounts of irrelevant data. Using the present invention it is possible to completely avoid the need for microarrays if the limited probe set(s) of the present invention are used with, e.g., digital optical chemistry arrays, ball bead arrays, beads (e.g., Luminex), multiplex PCR, quantitiative PCR, run-on assays, Northern blot analysis, or even, for protein analysis, e.g., Western blot analysis, 2-D and 3-D gel protein expression, MALDI, MALDI-TOF, fluorescence activated cell sorting (FACS) (cell surface or intracellular), enzyme linked immunosorbent assays (ELISA), chemiluminescence studies, enzymatic assays, proliferation studies or any other method, apparatus and system for the determination and/or analysis of gene expression that are readily commercially available.

The “molecular fingerprinting system” of the present invention may be used to facilitate and conduct a comparative analysis of expression in different cells or tissues, different subpopulations of the same cells or tissues, different physiological states of the same cells or tissue, different developmental stages of the same cells or tissue, or different cell populations of the same tissue against other diseases and/or normal cell controls. In some cases, the normal or wild-type expression data may be from samples analyzed at or about the same time or it may be expression data obtained or culled from existing gene array expression databases, e.g., public databases such as the NCBI Gene Expression Omnibus database.

As used herein, the term “differentially expressed” refers to the measurement of a cellular constituent (e.g., nucleic acid, protein, enzymatic activity and the like) that varies in two or more samples, e.g., between a disease sample and a normal sample. The cellular constituent may be on or off (present or absent), upregulated relative to a reference or down-regulated relative to the reference. For use with gene-chips or gene-arrays, differential gene expression of nucleic acids, e.g., mRNA or other RNAs (miRNA, siRNA, hnRNA, rRNA, tRNA, etc.) may be used to distinguish between cell types or nucleic acids. Most commonly, the measurement of the transcriptional state of a cell is accomplished by quantitative reverse transcriptase (RT) and/or quantitative reverse transcriptase-polymerase chain reaction (RT-PCR), genomic expression analysis, post-translational analysis, modifications to genomic DNA, translocations, in situ hybridization and the like.

For some disease states it is possible to identify cellular or morphological differences, especially at early levels of the disease state. The present invention avoids the need to identify those specific mutations or one or more genes by looking at modules of genes of the cells themselves or, more importantly, of the cellular RNA expression of genes from immune effector cells that are acting within their regular physiologic context, that is, during immune activation, immune tolerance or even immune anergy. While a genetic mutation may result in a dramatic change in the expression levels of a group of genes, biological systems often compensate for changes by altering the expression of other genes. As a result of these internal compensation responses, many perturbations may have minimal effects on observable phenotypes of the system but profound effects to the composition of cellular constituents. Likewise, the actual copies of a gene transcript may not increase or decrease, however, the longevity or half-life of the transcript may be affected leading to greatly increases protein production. The present invention eliminates the need of detecting the actual message by, in one embodiment, looking at effector cells (e.g., leukocytes, lymphocytes and/or sub-populations thereof) rather than single messages and/or mutations.

The skilled artisan will appreciate readily that samples may be obtained from a variety of sources including, e.g., single cells, a collection of cells, tissue, cell culture and the like. In certain cases, it may even be possible to isolate sufficient RNA from cells found in, e.g., urine, blood, saliva, tissue or biopsy samples and the like. In certain circumstances, enough cells and/or RNA may be obtained from: mucosal secretion, feces, tears, blood plasma, peritoneal fluid, interstitial fluid, intradural, cerebrospinal fluid, sweat or other bodily fluids. The nucleic acid source, e.g., from tissue or cell sources, may include a tissue biopsy sample, one or more sorted cell populations, cell culture, cell clones, transformed cells, biopies or a single cell. The tissue source may include, e.g., brain, liver, heart, kidney, lung, spleen, retina, bone, neural, lymph node, endocrine gland, reproductive organ, blood, nerve, vascular tissue, and olfactory epithelium.

The present invention includes the following basic components, which may be used alone or in combination, namely, one or more data mining algorithms; one or more module-level analytical processes; the characterization of blood leukocyte transcriptional modules; the use of aggregated modular data in multivariate analyses for the molecular diagnostic/prognostic of human diseases; and/or visualization of module-level data and results. Using the present invention it is also possible to develop and analyze composite transcriptional markers, which may be further aggregated into a single multivariate score.

The present inventors have recognized that current microarray-based research is facing significant challenges with the analysis of data that are notoriously “noisy,” that is, data that is difficult to interpret and does not compare well across laboratories and platforms. A widely accepted approach for the analysis of microarray data begins with the identification of subsets of genes differentially expressed between study groups. Next, the users try subsequently to “make sense” out of resulting gene lists using pattern discovery algorithms and existing scientific knowledge.

Rather than deal with the great variability across platforms, the present inventors have developed a strategy that emphasized the selection of biologically relevant genes at an early stage of the analysis. Briefly, the method includes the identification of the transcriptional components characterizing a given biological system for which an improved data mining algorithm was developed to analyze and extract groups of coordinately expressed genes, or transcriptional modules, from large collections of data.

The biomarker discovery strategy described herein is particularly well adapted for the exploitation of microarray data acquired on a global scale. Starting from 44,000 transcripts a set of 28 modules was defined that are composed of nearly 5000 transcripts. Sets of disease-specific composite expression vectors were then derived. Vector expression values (expression vectors) proved remarkably robust, as indicated by the excellent reproducibility obtained across microarray platforms. This finding is notable, since improving the reliability of microarray data is a prerequisite for the widespread use of this technology in clinical practice. Finally, expression vectors can in turn be combined to obtain unique multivariate scores, therefore delivering results in a form that is compatible with mainstream clinical practice. Interestingly, multivariate scores recapitulate global patterns of change rather than changes in individual markers. The development of such “global biomarkers” can be used for both diagnostic and pharmacogenomics fields.

In one example, twenty-eight transcriptional modules regrouping 4742 probe sets were obtained from 239 blood leukocyte transcriptional profiles. Functional convergence among genes forming these modules was demonstrated through literature profiling. The second step consisted of studying perturbations of transcriptional systems on a modular basis. To illustrate this concept, leukocyte transcriptional profiles obtained from healthy volunteers and patients were obtained, compared and analyzed. Further validation of this gene fingerprinting strategy was obtained through the analysis of a published microarray dataset. Remarkably, the modular transcriptional apparatus, system and methods of the present invention using pre-existing data showed a high degree of reproducibility across two commercial microarray platforms.

The present invention includes the implementation of a widely applicable, two-step microarray data mining strategy designed for the modular analysis of transcriptional systems. This novel approach was used to characterize transcriptional signatures of blood leukocytes, which constitutes the most accessible source of clinically relevant information.

As demonstrated herein, it is possible to determine, differential and/or distinguish between two disease based on two vectors even if the vector is identical (+/+) for two diseases—e.g. M1.3=53% down for both SLE and FLU because the composition of each vector can still be used to differentiate them. For example, even though the proportion and polarity of differentially expressed transcripts is identical between the two diseases for M1.3, the gene composition can still be disease-specific. The combination of gene-level and module-level analysis considerably increases resolution. Furthermore, it is possible to use 2, 3, 4, 5, 10, 15, 20, 25, 28 or more modules to differentiate diseases.

The term “gene” refers to a nucleic acid (e.g., DNA) sequence that includes coding sequences necessary for the production of a polypeptide (e.g.,), precursor, or RNA (e.g., mRNA). The polypeptide may be encoded by a full length coding sequence or by any portion of the coding sequence so long as the desired activity or functional property (e.g., enzymatic activity, ligand binding, signal transduction, immunogenicity, etc.) of the full-length or fragment is retained. The term also encompasses the coding region of a structural gene and the sequences located adjacent to the coding region on both the 5′ and 3′ ends for a distance of about 2 kb or more on either end such that the gene corresponds to the length of the full-length mRNA and 5′ regulatory sequences which influence the transcriptional properties of the gene. Sequences located 5′ of the coding region and present on the mRNA are referred to as 5′-untranslated sequences. The 5′-untranslated sequences usually contain the regulatory sequences. Sequences located 3′ or downstream of the coding region and present on the mRNA are referred to as 3′-untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “nucleic acid” refers to any nucleic acid containing molecule, including but not limited to, DNA, cDNA and RNA. In particular, the terms “a gene in Table X” refers to at least a portion or the full-length sequence listed in a particular table, as found hereinbelow. The gene may even be found or detected a genomic form, that is, it includes one or more intron(s). Genomic forms of a gene may also include sequences located on both the 5′ and 3′ end of the coding sequences that are present on the RNA transcript. These sequences are referred to as “flanking” sequences or regions. The 5′ flanking region may contain regulatory sequences such as promoters and enhancers that control or influence the transcription of the gene. The 3′ flanking region may contain sequences that influence the transcription termination, post-transcriptional cleavage, mRNA stability and polyadenylation.

As used herein, the term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the term “polymorphism” refers to the regular and simultaneous occurrence in a single interbreeding population of two or more alleles of a gene, where the frequency of the rarer alleles is greater than can be explained by recurrent mutation alone (typically greater than 1%).

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide protein) chain. The DNA sequence thus codes for the amino acid sequence.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the Tm of the formed hybrid, and the G:C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely related sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. Any probe used in the present invention may be labeled with any “reporter molecule,” so that it is detectable in any detection system, including, but not limited to enzyme (e.g., ELISA, as well as enzyme-based histochemical assays), fluorescent, radioactive, luminescent systems and the like. It is not intended that the present invention be limited to any particular detection system or label.

As used herein, the term “target,” refers to the region of nucleic acid bounded by the primers. Thus, the “target” is sought to be sorted out from other nucleic acid sequences. A “segment” is defined as a region of nucleic acid within the target sequence.

As used herein, the term “Southern blot” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (Sambrook et al., Molecular Cloning: A Laboratory Manual, Cold Spring Harbor Press, NY, pp 9.31-9.58, 1989).

As used herein, the term “Northern blot” refers to the analysis of RNA by electrophoresis of RNA on agarose gels, to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (Sambrook, et al., supra, pp 7.39-7.52, 1989).

As used herein, the term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

As used herein, the term “polymerase chain reaction” (“PCR”) refers to the method of K. B. Mullis (U.S. Pat. Nos. 4,683,195 4,683,202, and 4,965,188, hereby incorporated by reference), which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “real time PCR” as used herein, refers to various PCR applications in which amplification is measured during as opposed to after completion of the reaction. Reagents suitable for use in real time PCR embodiments of the present invention include but are not limited to TaqMan probes, molecular beacons, Scorpions primers or double-stranded DNA binding dyes.

As used herein, the terms “transcriptional upregulation,” “overexpression, and “overexpressed” refers to an increase in synthesis of RNA, by RNA polymerases using a DNA template. For example, when used in reference to the methods of the present invention, the term “transcriptional upregulation” refers to an increase of about 1 fold, 2 fold, 2 to 3 fold, 3 to 10 fold, and even greater than 10 fold, in the quantity of mRNA corresponding to a gene of interest detected in a sample derived from an individual predisposed to SLE as compared to that detected in a sample derived from an individual who is not predisposed to SLE. However, the system and evaluation is sufficiently specific to require less that a 2 fold change in expression to be detected. Furthermore, the change in expression may be at the cellular level (change in expression within a single cell or cell populations) or may even be evaluated at a tissue level, where there is a change in the number of cells that are expressing the gene. Changes of gene expression in the context of the analysis of a tissue can be due to either regulation of gene activity or relative change in cellular composition. Particularly useful differences are those that are statistically significant.

Conversely, the terms “transcriptional downregulation,” “underexpression” and “underexpressed” are used interchangeably and refer to a decrease in synthesis of RNA, by RNA polymerases using a DNA template. For example, when used in reference to the methods of the present invention, the term “transcriptional downregulation” refers to a decrease of least 1 fold, 2 fold, 2 to 3 fold, 3 to 10 fold, and even greater than 10 fold, in the quantity of mRNA corresponding to a gene of interest detected in a sample derived from an individual predisposed to SLE as compared to that detected in a sample derived from an individual who is not predisposed to such a condition or to a database of information for wild-type and/or normal control, e.g., fibromyalgia. Again, the system and evaluation is sufficiently specific to require less that a 2 fold change in expression to be detected. Particularly useful differences are those that are statistically significant.

Both transcriptional “upregulation”/overexpression and transcriptional “downregulation”/underexpression may also be indirectly monitored through measurement of the translation product or protein level corresponding to the gene of interest. The present invention is not limited to any given mechanism related to upregulation or downregulation of transcription.

The term “eukaryotic cell” as used herein refers to a cell or organism with membrane-bound, structurally discrete nucleus and other well-developed subcellular compartments. Eukaryotes include all organisms except viruses, bacteria, and bluegreen algae.

As used herein, the term “in vitro transcription” refers to a transcription reaction comprising a purified DNA template containing a promoter, ribonucleotide triphosphates, a buffer system that includes a reducing agent and cations, e.g., DTT and magnesium ions, and an appropriate RNA polymerase, which is performed outside of a living cell or organism.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

As used herein, the term “diagnosis” refers to the determination of the nature of a case of disease. In some embodiments of the present invention, methods for making a diagnosis are provided which permit determination of the infectious agents or agents that are the source of the infectious disease. In certain embodiments, the analysis of the present invention may be combined with one or more of the modules of co-pending patent application Ser. Nos. 60,748,884, 11,446,825 and ______, relevant portions incorporated herein by reference, for the determination of the nature of a disease condition, e.g., auto-immune diseases, auto-inflammatory diseases, cancer, transplant rejection, viral infection, bacterial infection, helminthic or parasitic infection and the like.

The present invention may be used alone or in combination with disease therapy to monitor disease progression and/or patient management. For example, a patient may be tested one or more times to determine the best course of treatment, determine if the treatment is having the intended medical effect, if the patient is not a candidate for that particular therapy and combinations thereof. The skilled artisan will recognize that one or more of the expression vectors may be indicative of one or more diseases and may be affected by other conditions, be they acute or chronic.

As used herein, the term “pharmacogenetic test” refers to an assay intended to study interindividual variations in DNA sequence related to, e.g., drug absorption and disposition (pharmacokinetics) or drug action (pharmacodynamics), which may include polymorphic variations in one or more genes that encode the functions of, e.g., transporters, metabolizing enzymes, receptors and other proteins.

As used herein, the term “pharmacogenomic test” refers to an assay used to study interindividual variations in whole-genome or candidate genes, e.g., single-nucleotide polymorphism (SNP) maps or haplotype markers, and the alteration of gene expression or inactivation that may be correlated with pharmacological function and therapeutic response.

As used herein, an “expression profile” refers to the measurement of the relative abundance of a plurality of cellular constituents. Such measurements may include, e.g., RNA or protein abundances or activity levels. The expression profile can be a measurement for example of the transcriptional state or the translational state. See U.S. Pat. Nos. 6,040,138, 5,800,992, 6,020135, 6,033,860, relevant portions incorporated herein by reference. The gene expression monitoring system, include nucleic acid probe arrays, membrane blot (such as used in hybridization analysis such as Northern, Southern, dot, and the like), or microwells, sample tubes, gels, beads or fibers (or any solid support comprising bound nucleic acids). See, e.g., U.S. Pat. Nos. 5,770,722, 5,874,219, 5,744,305, 5,677,195 and 5,445,934, relevant portions incorporated herein by reference. The gene expression monitoring system may also comprise nucleic acid probes in solution.

The gene expression monitoring system according to the present invention may be used to facilitate a comparative analysis of expression in different cells or tissues, different subpopulations of the same cells or tissues, different physiological states of the same cells or tissue, different developmental stages of the same cells or tissue, or different cell populations of the same tissue.

As used herein, the term “differentially expressed: refers to the measurement of a cellular constituent varies in two or more samples. The cellular constituent can be either up-regulated in the test sample relative to the reference or down-regulated in the test sample relative to one or more references. Differential gene expression can also be used to distinguish between cell types or nucleic acids. See U.S. Pat. No. 5,800,992, relevant portions incorporated herein by reference.

Therapy or Therapeutic Regimen: In order to alleviate or alter a disease state, a therapy or therapeutic regimen is often undertaken. A therapy or therapeutic regimen, as used herein, refers to a course of treatment intended to reduce or eliminate the affects or symptoms of a disease. A therapeutic regimen will typically comprise, but is not limited to, a prescribed dosage of one or more drugs or surgery. Therapies, ideally, will be beneficial and reduce the disease state but in many instances the effect of a therapy will have non-desirable effects as well. The effect of therapy will also be impacted by the physiological state of the sample.

As used herein, the term “pharmacological state” or “pharmacological status” refers to those samples that will be, are and/or were treated with one or more drugs, surgery and the like that may affect the pharmacological state of one or more nucleic acids in a sample, e.g., newly transcribed, stabilized or destabilized as a result of the pharmacological intervention. The pharmacological state of a sample relates to changes in the biological status before, during and/or after drug treatment and may serve a diagnostic or prognostic function, as taught herein. Some changes following drug treatment or surgery may be relevant to the disease state and/or may be unrelated side-effects of the therapy. Changes in the pharmacological state are the likely results of the duration of therapy, types and doses of drugs prescribed, degree of compliance with a given course of therapy, and/or un-prescribed drugs ingested.

Because each pathogen represents a unique combination of Pathogen Associated Molecular Patterns (PAMPs) interacting with specific pattern recognition receptors (PRRs), the present inventors determined if leukocytes isolated from the peripheral blood of patients with acute infections would carry unique transcriptional signatures, which would in turn permit pathogen discrimination. To test this hypothesis, gene expression patterns in blood leukocytes from patients with acute infections caused by four common human pathogens: (i) influenza A, an RNA virus; (ii) Staphylococcus aureus; and (iii) Streptococcus pneumoniae, two Gram-positive bacteria; and (iv) Escherichia coli, a Gram-negative bacterium were analyzed.

TABLE 2
Characteristics of 141 patients with acute infections, and 7 Healthy Controls.

| | | | | | | | |
|---|---|---|---|---|---|---|---|
| | | | | | Bacteria vs | E. coli vs | |
| Patient | Age | Ethnicity | Sex | Clinical disease | Virus | S. aureus | Antimicrobial therapy |

Set 1: E. coli (n = 29) Median age 2 m (2 wks-16 y)

| | | | | | | | |
|---|---|---|---|---|---|---|---|
| 12 | 5 m | Black | M | Bacteremia | Training | Training | Ceftriaxone |
| 13 | 5 m | White | F | UTI | Training | Training | Ceftriaxone |
| 31 | 3 m | Hispanic | F | UTI, bacteremia | Training | Training | Gentamicin |
| 34 | 16 y | White | F | Pyelonephritis | Test 1 | Training | Gentamicin |
| 48 | 2 m | White | M | UTI | Test 1 | Test 3 | Ampicillin, ceftriaxone |
| 57 | 3 m | Black | F | UTI, bacteremia | Test 1 | Training | Gentamicin |
| 74 | 4 m | Hispanic | F | UTI, bacteremia | Training | Training | Ceftriaxone |
| 82 | 2 m | Hispanic | M | UTI | Test 1 | Training | Ampicillin, ceftriaxone |
| 86 | 3 m | Hispanic | M | UTI | Training | Training | Ceftriaxone |
| 118 | 1.5 m | White | M | UTI | Test 1 | Test 3 | Test 1 & 2 |
| 120 | 1.5 m | Hispanic | M | UTI | Test 1 | Test 3 | Ampicillin, ceftriaxone |
| 133 | 2 m | Hispanic | M | UTI | Test 1 | Test 3 | Ceftriaxone |
| 139 | 1 m | Hispanic | M | UTI | Test 1 | Test 3 | Ampicillin, ceftriaxone |
| 148 | 8 y | Hispanic | F | UTI | Test 1 | Training | Ceftriaxone |
| 151 | 1.5 m | Hispanic | M | UTI | Test 1 | Test 3 | Ampicillin, gentamicin |
| 152 | 2.5 m | Black | M | Bacteremia, | Training | Training | Ceftriaxone, gentamicin |
| | | | | meningitis | | | |
| 154 | 2 m | Hispanic | M | UTI | Test 1 | Test 3 | Ceftriaxone |
| 161 | 1.7 m | Hispanic | M | UTI | Test 1 | Test 3 | Ampicillin, ceftriaxone |
| 168 | 3 m | White | F | UTI | Test 1 | Test 3 | Ceftriaxone |
| 171 | 3 m | Hispanic | F | UTI | Test 1 | Test 3 | Ceftriaxone |
| 175 | 0.5 m | Hispanic | F | UTI, bacteremia | Test 1 | Test 3 | Ceftriaxone |
| 180 | 1 m | Hispanic | M | UTI | Test 1 | Test 3 | Ampicillin, gentamicin |
| 183 | 1.5 m | Hispanic | M | UTI | Test 1 | Test 3 | Ampicillin, gentamicin, |
| | | | | | | | ceftriaxone |
| 184 | 0.5 m | White | F | UTI, bacteremia | Test 1 | Test 3 | Ampicillin, gentamicin |
| 188 | 1.5 m | White | M | UTI | Test 1 | Test 3 | Ampicillin, gentamicin, |
| | | | | | | | ceftriaxone |
| 197 | 1.25 m | White | M | UTI | Test 1 | Test 3 | Ampicillin, gentamicin |
| 219 | 5 m | White | F | UTI, bacteremia | Test 1 | Test 3 | Ceftriaxone |
| 222 | 3 m | Hispanic | F | UTI, bacteremia | Test 1 | Test 3 | Ceftriaxone, gentamicin |
| 229 | 4 m | Hispanic | F | UTI, bacteremia | Test 1 | Test 3 | Ceftriaxone |

Set 1: S. aureus (n = 32) Median age 7 y (3 m-18 y)

| | | | | | | | |
|---|---|---|---|---|---|---|---|
| 5 | 10 y | Hispanic | M | Osteomyelitis | Test 2 | Training | Cefazolin |
| 24 | 3 y | Black | M | Osteomyelitis | Test 2 | Test 3 | Vancomycin, Rifampin |
| 30 | 15 y | Black | M | Bacteremia | Test 2 | Test 3 | Vancomycin |
| 40 | 12 y | White | M | Osteomyelitis, | Test 2 | Test 3 | Cefazolin |
| | | | | Bacteremia | | | |
| 43 | 7 y | Black | M | Hip abscess, | Test 2 | Test 3 | Vancomycin, Rifampin |
| | | | | Bacteremia | | | |
| 62 | 2 y | White | M | Osteomyelitis | Test 2 | Training | Clindamycin |
| 66 | 3 m | Black | F | Pneumonia | Test 2 | Training | Vancomycin, Gentamicin |
| 67 | 7 y | White | F | Osteomyelitis, | Test 2 | Training | Vancomycin, Rifampin |
| | | | | Bacteremia | | | |
| 69 | 9 mo | Hispanic | M | Lung abscess | Test 2 | Training | Vancomycin, Cefazolin |
| 70 | 15 m | White | F | Abscess | Test 2 | Training | Vancomycin |
| 84 | 18 y | Black | F | Abscess | Test 2 | Test 3 | Cefazolin |
| 88 | 11 m | Hispanic | M | Osteomyelitis, | Test 2 | Training | Vancomycin |
| | | | | bacteremia | | | |
| 89 | 4 mo | Black | F | Abscess | Test 2 | Training | Clindamycin |
| 90 | 8 mo | Black | M | Septic arthritis | Test 2 | Training | Oxacillin |
| 150 | 9 y | Black | F | Osteomyelitis, | Test 2 | Test 3 | Vancomycin, Rifampin |
| | | | | Bacteremia | | | |
| 179 | 12 y | White | M | Endocarditis, | Test 2 | Test 3 | Oxacillin, Gentamicin, |
| | | | | Bacteremia. | | | Rifampin |
| 205 | 7 yo | Hispanic | M | Pneumonia, | Test 2 | Test 3 | Vancomycin |
| | | | | Bacteremia | | | |
| 206 | 1 y | Hispanic | F | Abscess | Test 2 | Test 3 | Clindamycin |
| 208 | 10 y | White | F | Osteomyelitis, | Test 2 | Test 3 | Vancomycin, Clindamycin, |
| | | | | Bacteremia, | | | Rifampin |
| | | | | pneumonia | | | |
| 216 | 10 y | Hispanic | F | Osteomyelitis, | Test 2 | Training | Vancomycin, Rifampin |
| | | | | Bacteremia | | | |
| 220 | 11 y | Hispanic | M | Osteomyelitis, | Test 2 | Test 3 | Cefazolin, Rifampin |
| | | | | Bacteremia | | | |
| 221 | 6 y | Black | F | Osteomyelitis, | Test 2 | Test 3 | Vancomycin, Rifampin |
| | | | | Bacteremia | | | |
| 224 | 10 y | White | M | Osteomyelitis, | Test 2 | Test 3 | Oxacillin, Rifampin |
| | | | | Bacteremia | | | |
| 241 | 10 m | Black | F | Pneumonia, | Test 2 | Test 3 | Vancomycin, Rifampin |
| | | | | Bacteremia | | | |
| 242 | 13 m | Black | M | Abscess, | Test 2 | Test 3 | Clindamycin |
| | | | | Bacteremia | | | |
| 258 | 8 y | White | F | Osteomyelitis, | Test 2 | Test 3 | Cefazolin |
| | | | | Bacteremia | | | |
| 262 | 13 y | Hispanic | M | Abscess, | Test 2 | Test 3 | Clindamycin |
| | | | | Bacteremia | | | |
| 264 | 13 y | Black | M | Septic arthritis | Test 2 | Test 3 | Vancomycin, cefazolin, |
| | | | | | | | gentamicin |
| 271 | 13 y | Black | M | Osteomyelitis | Test 2 | Test 3 | Clindamycin |
| 281 | 3 y | White | F | Osteomyelitis | Test 2 | Test 3 | Clindamycin |
| 315 | 3 y | Hispanic | F | Cellulitis | Test 2 | Test 3 | Vancomycin |
| 374 | 21 m | Black | M | Septic arthritis | | | Vancomycin, Clindamycin |
| | | | | Bacteremia | | | |

Set 1: S. pneumoniae (n = 16) Median age 1.5 y (2 m-16 y)

| | | | | | | | |
|---|---|---|---|---|---|---|---|
| 9 | 4 m | White | M | Abscess | Training | N/A | Cefazolin |
| 25 | 2 m | Hispanic | M | Meningitis | Training | N/A | Ampicillin, Ceftriaxone |
| 41 | 23 m | White | F | Pneumonia, | Training | N/A | Ceftriaxone |
| | | | | Empyema | | | |
| 64 | 10 m | White | F | Meningitis, | Test 1 | N/A | Ceftriaxone, vancomycin |
| | | | | bacteremia | | | |
| 96 | 16 m | Hispanic | M | Pneumonia, | Training | N/A | Ceftriaxone, Azithromycin |
| | | | | Empyema | | | |
| 113 | 7 m | Hispanic | F | Septic arthritis | Training | N/A | Ceftriaxone, clindamycin |
| 155 | 3 m | Hispanic | M | Meningitis | Training | N/A | Ceftriaxone, Vancomycin |
| 261 | 13 y | White | M | Meningitis | Test 1 | N/A | Ceftriaxone, vancomycin |
| 268 | 3 y | Hispanic | M | Empyema | Test 1 | N/A | Ceftriaxone, clindamycin |
| 265 | 2 y | White | F | Empyema | Test 1 | N/A | Ceftriaxone, vancomycin |
| 277 | 16 y | White | M | Empyema | Test 1 | N/A | Ceftriaxone, vancomycin |
| 287 | 3 y | White | F | Pneumonia, | Test 1 | N/A | Ceftriaxone, vancomycin |
| | | | | bacteremia | | | |
| 289 | 2 y | Hispanic | M | Empyema | Test 1 | N/A | Ceftriaxone, vancomycin |
| 338 | 12 m | White | M | Meningitis | | | Ceftriaxone, vancomycin |
| 339 | 2.5 y | White | M | Mastoiditis | | | Ceftriaxone |
| 388 | 6 m | White | M | Meningitis | | | Ceftriaxone, Vancomycin, |
| | | | | | | | Rifampin |

Set 1: Influenza A (n = 18) Median age 14 m (3 wks-36 y)

| 55 | 11 m | Hispanic | M | Respiratory | Training | N/A | Cefuroxime |
|---|---|---|---|---|---|---|---|
| | | | | distress | | | |
| 87 | 19 m | White | F | Fever, Hypoxia | Training | N/A | Cefuroxime |
| 92 | 1 m | Hispanic | F | Fever | Training | N/A | Ampicillin, Ceftriaxone |
| 95 | 4 y | Hispanic | M | Fever | Test 1 & 2 | N/A | None |
| 101 | 4 m | Hispanic | M | Fever, URI | Training | N/A | Cefuroxime, Oseltamivir |
| | | | | Seizures, Fever, | | | |
| 104 | 17 m | Hispanic | M | Respiratory | Training | N/A | Ceftriaxone |
| | | | | failure | | | |
| 105 | 4 y | Hispanic | F | Fever, | Test 1 & 2 | N/A | Ceftriaxone, Aciclovir, |
| | | | | Encephalopathy | | | Oseltamivir |
| 107 | 1.5 m | Asian | M | Fever, Lethargy | Training | N/A | Ampicillin, Ceftriaxone |
| 108 | 5 m | Hispanic | M | Fever | Training | N/A | Ceftriaxone |
| 112 | 1 m | Hispanic | M | Fever, URI | Test 1 & 2 | N/A | Ampicillin, Gentamycin |
| 114 | 18 m | Black | F | Respiratory | Training | N/A | Cefuroxime, Oseltamivir |
| | | | | distress, fever | | | |
| 115 | 20 m | White | M | Seizures | Training | N/A | Amoxicillin |
| 116 | 2 y | White | M | Fever, URI | Test 1 & 2 | N/A | Cefuroxime, Clindamycin |
| 117 | 24 y | White | F | Fever | Test 1 & 2 | N/A | None |
| 128 | 11 m | Hispanic | F | Fever, Hypoxia | Training | N/A | Cefuroxime |
| 132 | 6 m | White | M | Respiratory | Training | N/A | Oxacillin, Tobramycin |
| | | | | distress, fever | | | |
| 259 | 3 m | Hispanic | F | Pneumonia | Test 1 & 2 | N/A | None |
| 266 | 36 y | White | F | Fever, cough | Test 1 & 2 | N/A | None |
| Patient | Age | Ethnicity | Sex | Clinical disease | Analysis | Platform | Antimicrobial therapy |

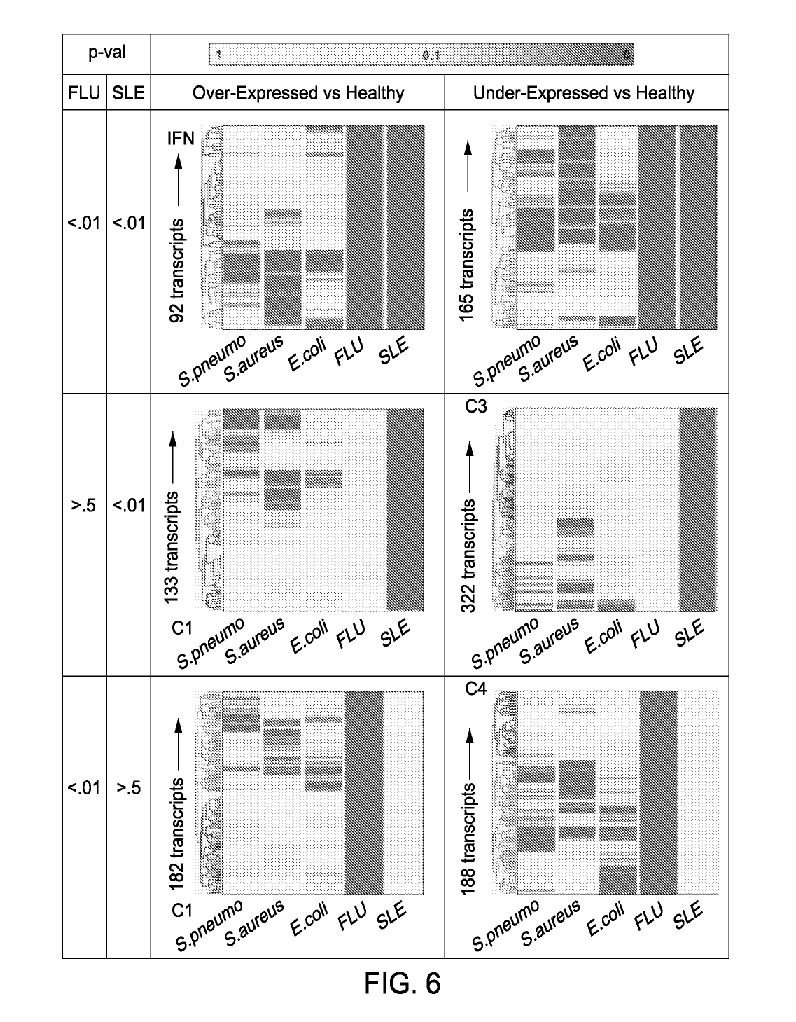

Set 2: Influenza (n = 18) Median age = 11 m (2 wk-13 y)

| | | | | | | | |
|---|---|---|---|---|---|---|---|
| 311 | 0.1 y | Hispanic | M | Influenza B | FIG. 6c | Illumina | Ampicillin + Ceftriaxone |
| | | | | Fever, URI | | Sentrix Hu6 | |
| 320 | 0.04 y | Hispanic | F | Influenza B | FIG. 6c | Illumina | Ampicillin + Gentamicin |
| | | | | Fever, URI | | Sentrix Hu6 | |
| 517 | 0.5 y | Hispanic | F | Influenza A | FIG. 6a | Affymetrix | None |
| | | | | Pneumonia | FIG. 6b | U133plus2 | |
| 519 | 0.13 y | Hispanic | F | Influenza A | FIG. 6c | Illumina | None |
| | | | | Fever | | Sentrix Hu6 | |
| 524 | 6 y | Hispanic | M | Influenza A | FIG. 6a | Affymetrix | None |
| | | | | Fever | | U133plus2 | |
| 527 | 0.13 y | Black | M | Influenza A | FIG. 6c | Illumina | Ampicillin + Ceftriaxone |
| | | | | Fever | | Sentrix Hu6 | |
| 530 | 0.38 y | Hispanic | M | Influenza A | FIG. 6c | Illumina | None |
| | | | | Fever, Seizure | | Sentrix Hu6 | |
| 532 | 0.08 y | Hispanic | F | Influenza A | FIG. 6a | Affymetrix | Ampicillin + Gentamicin |
| | | | | Fever, Cough | FIG. 6b | U133plus2 | |
| 533 | 11 y | Caucasian | M | Influenza B | FIG. 6a | Affymetrix | None |
| | | | | Fever, Cough | FIG. 6b | U133plus2 | |
| 536 | 2 y | Hispanic | F | Influenza A | FIG. 6a | Affymetrix | None |
| | | | | Fever, Cough | FIG. 6b | U133plus2 | |
| 540 | 0.08 y | Hispanic | M | Influenza A | FIG. 6a | Affymetrix | Ampicillin + Gentamicin |
| | | | | Fever, Cough | FIG. 6b | U133plus2 | |
| 542 | 0.04 y | Hispanic | F | Influenza A | FIG. 6c | Illumina | Ampicillin + Gentamicin |
| | | | | Fever | | Sentrix Hu6 | |
| 547 | 1.33 y | Black | F | Influenza A | FIG. 6a | Affymetrix | Ceftriaxone + Oseltamivir |
| | | | | Encephalitis | | U133plus2 | |
| 549 | 13 y | Hispanic | F | Influenza B | FIG. 6a | Affymetrix | Ceftriaxone + Vancomycin + Oseltamivir |
| | | | | Fever, Syncope | | U133plus2 | |
| 553 | 1.5 y | Caucasian | F | Influenza A | FIG. 6a | Affymetrix | Oseltamivir |
| | | | | Fever, URI | FIG. 6b | U133plus2 | |
| 556 | 3.5 y | Caucasian | F | Influenza A | FIG. 6c | Illumina | Ceftriaxone + Oseltamivir |
| | | | | Fever, Seizure | | Sentrix Hu6 | |
| 560 | 10 y | Black | F | Influenza B | FIG. 6c | Illumina | Acyclovir |
| | | | | Encephalitis | | Sentrix Hu6 | |
| 567 | 2 y | Hispanic | F | Influenza B | FIG. 6a | Affymetrix | None |
| | | | | Fever, URI | FIG. 6b | U133plus2 | |

Set 2: S. aureus (n = 19) Median age = 7.5 y (0.08-14 y)

| | | | | | | | |
|---|---|---|---|---|---|---|---|
| 305 | 4.5 y | Hispanic | F | MSSA | FIG. 6a | Affymetrix | Cefazolin |
| | | | | Bacteremia, | | U133plus2 | |
| | | | | Suppurative | | | |
| | | | | Arthritis, | | | |
| | | | | Osteomyelitis | | | |
| 308 | 12 y | Black | F | MSSA | FIG. 6a | Affymetrix | Oxacillin + Clindamycin |
| | | | | Disseminated | FIG. 6b | U133plus2 | |
| | | | | with Pneumonia | | | |
| 369 | 14 y | Black | M | MRSA | FIG. 6a | Affymetrix | Vancomycin, Rifampin |
| | | | | Disseminated | | U133plus2 | |
| 372 | 14 y | Caucasian | M | MRSA | FIG. 6a | Affymetrix | Vancomycin, Rifampin |
| | | | | Bacteremia, | | U133plus2 | |
| | | | | Osteomyelitis | | | |
| 374 | 1.75 | Black | M | MRSA | FIG. 6a | Affymetrix | Vancomycin |
| | | | | Bacteremia, | | U133plus2 | |
| | | | | Suppurative | | | |
| | | | | Arthritis | | | |
| 380 | 7.5 y | Black | M | MRSA | FIG. 6a | Affymetrix | Clindamycin |
| | | | | Osteomyelitis, | | U133plus2 | |
| | | | | Suppurative | | | |
| | | | | Arthritis | | | |
| 458 | 12 y | Black | M | MRSA | FIG. 6c | Illumina | Vancomycin + Rifampin + Linezolid |
| | | | | Disseminated | | Sentrix Hu6 | |
| 459 | 10 y | Caucasian | F | MSSA | FIG. 6c | Illumina | Oxacillin + Rifampin |
| | | | | Osteomyelitis, | | Sentrix Hu6 | |
| | | | | Suppurative | | | |
| | | | | Arthritis | | | |
| 465 | 13 y | Caucasian | M | MRSA | FIG. 6c | Illumina | Vancomycin |
| | | | | Osteomyelitis, | | Sentrix Hu6 | |
| | | | | Suppurative | | | |
| | | | | Arthritis, | | | |
| | | | | Bacteremia | | | |
| 466 | 0.5 y | Black | M | MRSA | FIG. 6c | Illumina | Clindamycin |
| | | | | SST Abscess | | Sentrix Hu6 | |
| 472 | 0.08 y | Caucasian | M | MSSA | FIG. 6c | Illumina | Cefazolin |
| | | | | SST Abscess | | Sentrix Hu6 | |
| 475 | 1.33 y | Black | M | MSSA | FIG. 6c | Illumina | Nafcillin |
| | | | | Suppurative | | Sentrix Hu6 | |
| | | | | Arthritis | | | |
| 477 | 6 y | Black | M | MRSA | FIG. 6c | Illumina | Clindamycin + Rifampin |
| | | | | Bacteremia, | | Sentrix Hu6 | |
| | | | | Suppurative | | | |
| | | | | Arthritis | | | |
| 480 | 12 y | Caucasian | M | MSSA | FIG. 6c | Illumina | Clindamycin + Doxycicline |
| | | | | Bacteremia | | Sentrix Hu6 | |
| 489 | 1.08 y | Caucasian | M | MRSA | FIG. 6c | Illumina | Clindamycin |
| | | | | SST Abscess | | Sentrix Hu6 | |
| 522 | 9.5 y | Black | F | MRSA | FIG. 6c | Illumina | Vancomycin + Rifampin |
| | | | | Bacteremia, | | Sentrix Hu6 | |
| | | | | Osteomyelitis | | | |
| 529 | 1.75 | Black | M | MRSA | FIG. 6c | Illumina | Vancomycin + Rifampin |
| | | | | Bacteremia, | | Sentrix Hu6 | |
| | | | | Pneumonia | | | |
| 535 | 0.58 y | Other | F | MSSA | FIG. 6c | Illumina | Cefazolin |
| | | | | Suppurative | | Sentrix Hu6 | |
| | | | | arthritis | | | |
| 537 | 9 y | Black | F | MSSA | FIG. 6c | Illumina | Oxacillin |
| | | | | Bacteremia, | | Sentrix Hu6 | |
| | | | | Osteomyelitis, | | | |
| | | | | Suppurative | | | |
| | | | | Arthritis | | | |

Set 2: S. pneumoniae (n = 9) Median age = 2.5 y (1.3-16 y)

| | | | | | | | |
|---|---|---|---|---|---|---|---|
| 96 | 1.33 y | Hispanic | M | Pneumonia, | FIG. 6a | Affymetrix | Ceftriaxone + Azithromycin |
| | | | | Empyema | FIG. 6b | U133plus2 | |
| 265 | 2.2 y | Caucasian | F | Pneumonia, | FIG. 6a | Affymetrix | Ceftriaxone + Vancomycin |
| | | | | Empyema | FIG. 6b | U133plus2 | |
| 268 | 3 y | Hispanic | M | Pneumonia, | FIG. 6a | Affymetrix | Ceftriaxone |
| | | | | Empyema | FIG. 6b | U133plus2 | |
| 277 | 16 y | Caucasian | M | Pneumonia, | FIG. 6a | Affymetrix | Ceftriaxone + Clindamycin |
| | | | | Empyema | FIG. 6b | U133plus2 | |
| 287 | 3.2 y | Caucasian | F | Pneumonia, | FIG. 6a | Affymetrix | Ceftriaxone |
| | | | | Bacteremia | FIG. 6b | U133plus2 | |
| 289 | 2.5 y | Hispanic | M | Pneumonia, | FIG. 6a | Affymetrix | Ceftriaxone |
| | | | | Empyema | FIG. 6b | U133plus2 | |
| 471 | 2 y | Caucasian | F | Bacteremia, | FIG. 6c | Illumina | Vancomycin + Ceftriaxone |
| | | | | Meningitis | | Sentrix Hu6 | |
| 473 | 2.5 y | Hispanic | M | Bacteremia, | FIG. 6c | Illumina | Ceftriaxone |
| | | | | Pneumonia | | Sentrix Hu6 | |
| 523 | 3 y | Hispanic | M | Suppurative | FIG. 6c | Illumina | Cefazolin |
| | | | | Arthritis | | Sentrix Hu6 | |

TABLE 3
SLE Patient demographics
SLE patients (n = 11) 13 y (9-17 y)

| | | | | |
|---|---|---|---|---|
| SLE 87 | 11 y | White | F | N/A |
| SLE 85 | 16 y | Black | F | N/A |
| SLE 79 | 10 y | Hispanic | F | N/A |
| SLE 76 | 15 y | Black | F | N/A |
| SLE 66 | 17 y | Hispanic | F | N/A |
| SLE 57 | 12 y | Black | M | N/A |
| SLE 48 | 14 y | White | F | N/A |
| SLE 45 | 9 y | Black | F | N/A |
| SLE 107 | 14 y | Black | F | N/A |
| SLE 27 | 13 y | Black | F | N/A |
| SLE 19 | 9 y | Black | M | N/A |

Healthy controls (n = 17) 4.5 y (4 m-17 y)

| | | | | |
|---|---|---|---|---|
| INF 20N | 11 m | Hispanic | M | Healthy |
| INF 19N | 4 m | Hispanic | M | Healthy |
| INF 27N | 10 m | White | M | Healthy |
| INF 25N | 11 m | Hispanic | F | Healthy |
| INF 204 | 2 y | White | M | Healthy |
| INF 7N | 19 m | Black | F | Healthy |
| INF 391 | 18 m | White | F | Healthy |
| INF 392 | 10 m | Black | M | Healthy |
| H 162 | 15 y | Black | F | Healthy |
| HJM | 13 y | Hispanic | F | Healthy |
| HJC | 8 y | Hispanic | M | Healthy |
| HBW | 14 y | White | F | Healthy |
| H45 | 12 y | Black | F | Healthy |
| H42 | 9 y | Hispanic | M | Healthy |
| H36 | 7 y | White | F | Healthy |
| H28 | 11 y | Hispanic | F | Healthy |
| H37 | 8 y | White | F | Healthy |

TABLE 4
List of the 35 classifier genes distinguishing influenza A from
bacterial infections. Genes are grouped functionally based on
ontologies and levels of significance are
shown. Full details are available in Supplementary Table 3.
Influenza > Bacteria
Response to virus

| | |
|---|---|
| cig5 | 1.46E−05 |
| DNAPTP6 | 4.52E−06 |
| IFI27 | 4.52E−06 |
| IFI35 | 0.00033 |
| IFI44 | 0.00023 |
| IFI44 | 0.00015 |
| OAS1 | 6.52E−05 |

Immune response

| | |
|---|---|
| BST2 | 4.08E−05 |
| G1P2 | 0.000101 |
| LY6E | 8.28E−06 |
| MX1 | 6.52E−05 |

Anti-apoptosis

| | |
|---|---|
| SON | 0.00067 |

Cell growth and/or maintenance

| | |
|---|---|
| TRIM14 | 4.08E−05 |

Miscellaneous

| | |
|---|---|
| APOBEC3C | 2.35E−07 |
| C1orf29 | 0.00015 |
| FLJ20035 | 4.08E−05 |
| FLJ38348 | 0.00128 |
| HSXIAPAF1 | 4.52E−06 |
| KIAA0152 | 2.48E−05 |
| PHACTR2 | 9.34E−08 |
| USP18 | 1.46E−05 |
| ZBP1 | 5.41E−07 |

Bacteria > Influenza
Translational elongation

| | |
|---|---|
| EEF1G | 4.52E−06 |
| EEF1G | 2.35E−06 |

Regulation of translational initiation

| | |
|---|---|
| EIF3S5 | 9.34E−08 |
| EIF3S7 | 2.35E−07 |
| EIF4B | 1.16E−06 |

Protein biosynthesis

| | |
|---|---|
| QARS | 5.41E−07 |
| RPL31 | 4.52E−06 |
| RPL4 | 2.35E−07 |

Regulation of transcription

| | |
|---|---|
| PFDN5 | 5.41E−07 |

Cell adhesion

| | |
|---|---|
| CD44 | 2.35E−07 |

Metabolism

| | |
|---|---|
| HADHA | 4.08E−05 |
| PCBP2 | 9.34E−08 |

Miscellaneous

| | |
|---|---|
| dJ507I15.1 | 6.52E−05 |

TABLE 5
List of the 30 classifier genes distinguishing S. aureus from E. coli
infections. Genes are grouped functionally based on ontologies and
levels of significance are shown. Full details are available in
Supplementary Table 6.
S. aureus > E. coli
Signal Transduction

| | |
|---|---|
| CXCL1 | 0.00106 |
| JAG1 | 0.00158 |
| RGS2 | 0.00027 |

Metabolism

| | |
|---|---|
| GAPD | 0.00044 |
| PPIB | 0.00044 |
| PSMA7 | 0.00106 |
| MMP9 | 0.00837 |
| p44S10 | 0.00158 |

ProteinTargeting

| | |
|---|---|
| TRAM2 | 0.00384 |

Intracellular Protein Transport

| | |
|---|---|
| SEC24C | 4.92E−05 |

Miscellaneous

| | |
|---|---|
| ACTG1 | 0.00622 |
| CGI-96 | 0.00454 |
| MGC2963 | 0.00158 |
| STAU | 4.92E−05 |
| STAU | 4.92E−05 |

E. coli > S. aureus
Intracellular signaling

| | |
|---|---|
| RASA1 | 1.20E−05 |
| SNX4 | 4.92E−05 |

Regulation of translational initiation

| | |
|---|---|
| AF1Q | 0.00106 |

Regulation of transcription

| | |
|---|---|
| SMAD2 | 0.00044 |

Cell adhesion

| | |
|---|---|
| JUP | 0.00158 |

Metabolism

| | |
|---|---|
| PP | 4.92E−05 |
| MAN1C1 | 0.00016 |

Miscellaneous

| | |
|---|---|
| FLJ10287 | 4.92E−05 |
| FLJ20152 | 0.00622 |
| LRRN3 | 1.20E−05 |
| LRRN3 | 0.00027 |
| SGPP1 | 0.00158 |
| UBAP2L | 2.12E−06 |

Patient characteristics. The PBMCs from 29 patients with E. coli infections, 51 patients with S. aureus infections, 25 patients with S. pneumoniae infections and 36 patients with influenza A infections. We chose young patients because of fewer concomitant diseases and therapies than in older adults. Patients with underlying immunosuppression, receiving immunomodulatory therapy including corticosteroids, or with significant chronic medical problems were excluded. The median (range) duration of hospitalization at the time of blood draw was 3 days (0-9 days) and the median (range) duration of symptoms was 6 days (2-22 days). The clinical diagnoses included acute respiratory infections, bacteremia, localized abscesses, bone and joint infections, urinary tract infections and meningitis (Table 1). Patients were treated according to standard hospital protocols and, as such, antimicrobial therapy was promptly initiated in the emergency department.

Step-wise data analysis strategy. To determine whether blood leukocytes isolated from patients with acute infections carry gene expression signatures that allow discrimination between pathogen type, a step-wise analysis was conducted: (1) Statistical group comparison: differentially expressed genes were identified in pair-wise comparisons using non-parametric Mann-Whitney test. Hierarchical clustering ordered the genes according to their expression levels, revealing reciprocal patterns of expression between the two groups. (2) Sample classification: genes capable of discriminating two groups of patients, i.e. classifiers, were identified through comparison of patient groups of comparable age range and treated with similar classes of antimicrobials (training set). These genes were then evaluated within the same set of patients in a leave-one-out cross-validation scheme. (3) Independent validation of classifier genes: the same genes were tested for their ability to classify an independent group of patients (test set). The patients included in the training sets used to for the identification of the classifier genes were selected very carefully in order to avoid potential confounding factors. After that careful selection, the classifier genes (also described as transcriptional markers) were then evaluated in a new group of patients that was heterogeneous, and therefore more representative of a realistic clinical setting (test set). (4) Independent validation across microarray platforms: the results were then further validated in another independent set of patients using a different microarray platform (Illumina BeadChips).

Transcriptional signatures discriminate patients with influenza A infection from those with bacterial infections. To identify genes differentially expressed between samples from patients with either influenza or bacterial infections, 11 patients with influenza A infections and 12 patients with E. coli or S. pneumoniae infections were selected as a training set on the basis of similar age groups and antibiotic class treatment. There were no significant differences between the influenza A and the bacterial infection training groups in median age [range] (11 months [1-20 months] vs. 4 months [2 months-23 months]; P=0.22); or days of hospitalization prior to sample collection (2 days [1-2 days] vs. 2.5 days [2-5 days], P=0.06). All 11 patients with influenza A infections were receiving □-lactam antibiotics, as compared with 10 of 12 in the bacterial infection group (P=0.16). There were no statistically significant differences in the relative proportions of neutrophils, lymphocytes and monocytes in PBMCs from the two groups (Supplementary Table 1).

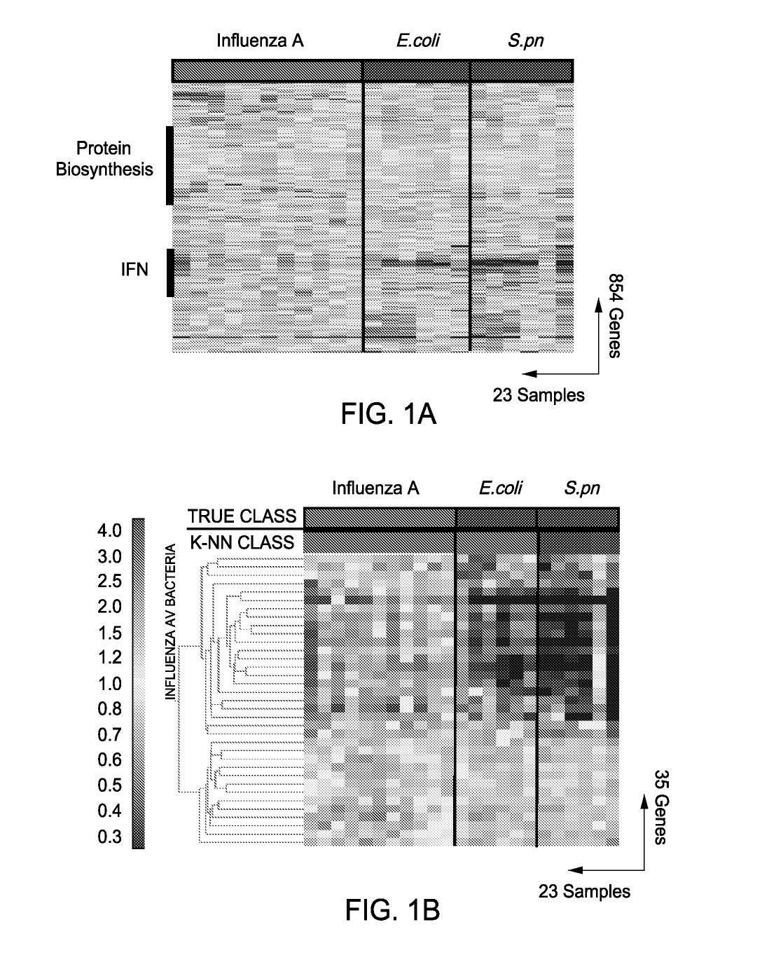

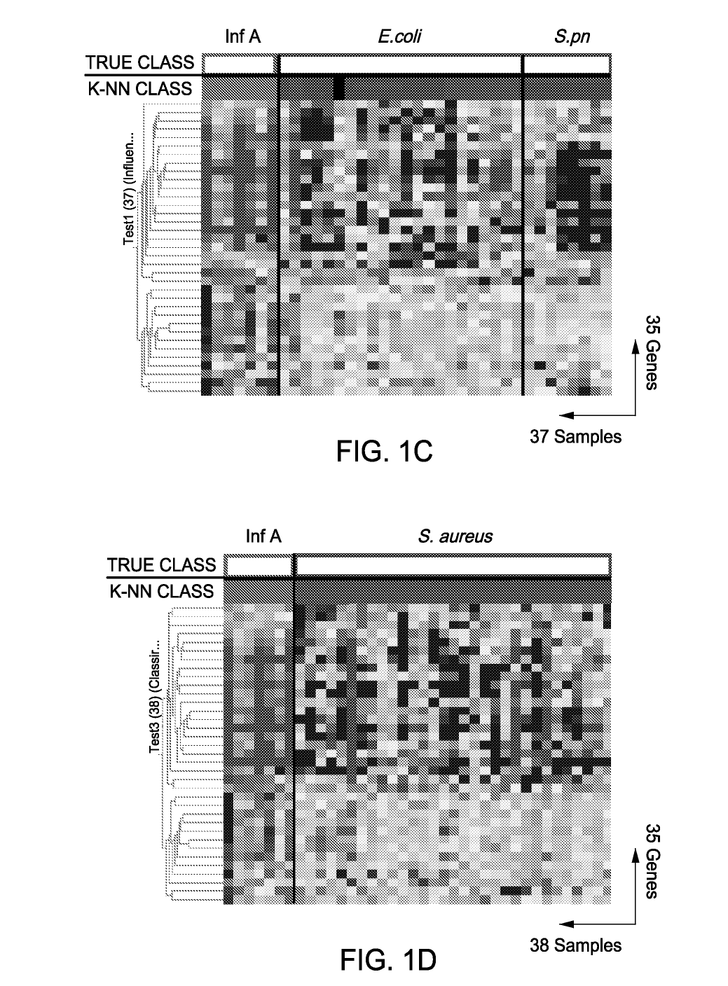

Statistical group comparisons of patients with influenza A and those with bacterial infections yielded 854 differentially expressed genes (P<0.01) (Supplementary Table 2), of which 394 were relatively over-expressed in influenza A infections, while 460 were over-expressed in bacterial infections. Patients with influenza A displayed a prominent type I interferon (IFN) signature (FIG. 1a), including genes coding for antiviral molecules such as myxovirus resistance genes (MX1, MX2); 2′-5′-oligoadenylate synthetases (OAS1, OAS2); GBP1 (Guanylate Binding Protein 1); and CIG5 (viperin, virus inhibitory protein, endoplasmic reticulum-associated, interferon-inducible). Genes regulating transcription and translation represent up to 25% of the 460 probe sets expressed at higher levels in the bacterial infection group.

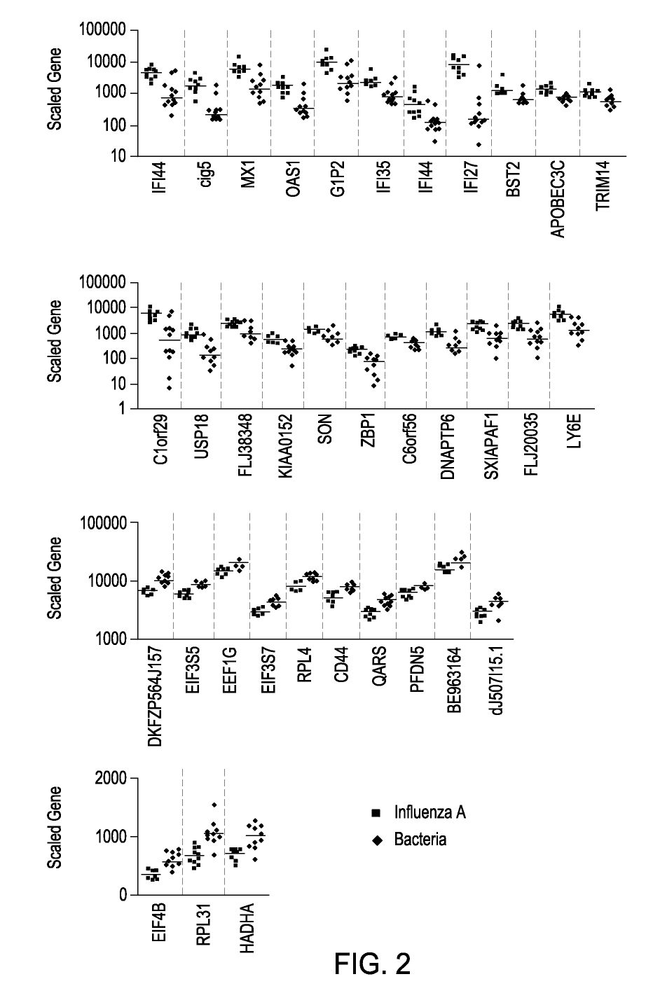

The k-NN algorithm identified 35 genes that discriminated patients with acute influenza infection from acute bacterial infections (FIG. 2, Table 2, and Supplementary Table 3). Leave-one-out cross-validation of this training set correctly classified 21 of the 23 samples (91% accuracy) to either the influenza A or the bacterial infection groups (FIG. 1b).

The ability of the identified classifier genes to discriminate influenza A from the bacterial infections was then validated with independent sets of samples (test sets). The first test set of patients included seven new patients with influenza A, and 30 patients with bacterial infections (seven with S. pneumoniae and 23 with E. coli infections). Patients were included in the test set without regard to age or type of antibiotic treatment (age [range]; influenza A, 4 years [3 weeks-36 years]; E. coli, 2 month [2 weeks-16 years]). Predictor genes correctly classified 35 of the 37 samples (95% accuracy) (FIG. 1c). One sample (INF48) was misclassified and one sample was of indeterminate classification (INF120).

The 35 classifier genes were then evaluated in a second test set, consisting of 7 patients with influenza A infection and 31 patients with S. aureus infection, yielding 87% accuracy in discrimination (FIG. 1d). Test sets were again selected without regard to age or type of antibiotic treatment (age [range]; influenza A, 4 years [3 weeks-36 years]; S. aureus, 7 years [3 months—15 years]). Five S. aureus samples were misclassified (INF62, INF70, INF89, INF221 and INF242).

About one-third of the patients with bacterial infection displayed elevated expression levels of interferon-related genes. This signature, however, had limited effects on classification outcomes, because samples obtained from patients with bacterial infections lacked the reciprocal expression signature characteristic of influenza infection (under-expressed genes in influenza compared to bacterial infection) and also in part because expression levels of interferon-inducible genes were lower in the context of bacterial infections (FIG. 1c). Elevated levels of expression of interferon-inducible genes may be attributed to a response to the documented bacterial infection itself [1,2], or an undiagnosed or preceding viral infection. Thus, transcriptional signatures of host response to influenza infection and bacterial infection can be identified. These signatures permit the discrimination between these causative agents.

Transcriptional signatures discriminate patients with E. coli infections from those with S. aureus infections. To identify genes differentially expressed between patients with E. coli and S. aureus infections, ten patients per group were selected as a training set. There were no significant differences between the E. coli and the S. aureus infection training groups in median age [range] (2 months [3.5 months-16 years] vs. 12 months [4 months-10 years]; P=0.06). Each group included 6 patients treated with β-lactam antibiotics and 4 with other antibiotic classes. Total peripheral leukocyte counts and the relative proportions of the peripheral blood cell types between the two groups were not significantly different (Supplementary Table 1). The median number of days of hospitalization prior to sample collection was 2 days for the E. coli group, and 4 days for the S. aureus group (P=0.01), a significant difference which may be accounted for by the time interval typically required for definitive microbiological diagnosis.

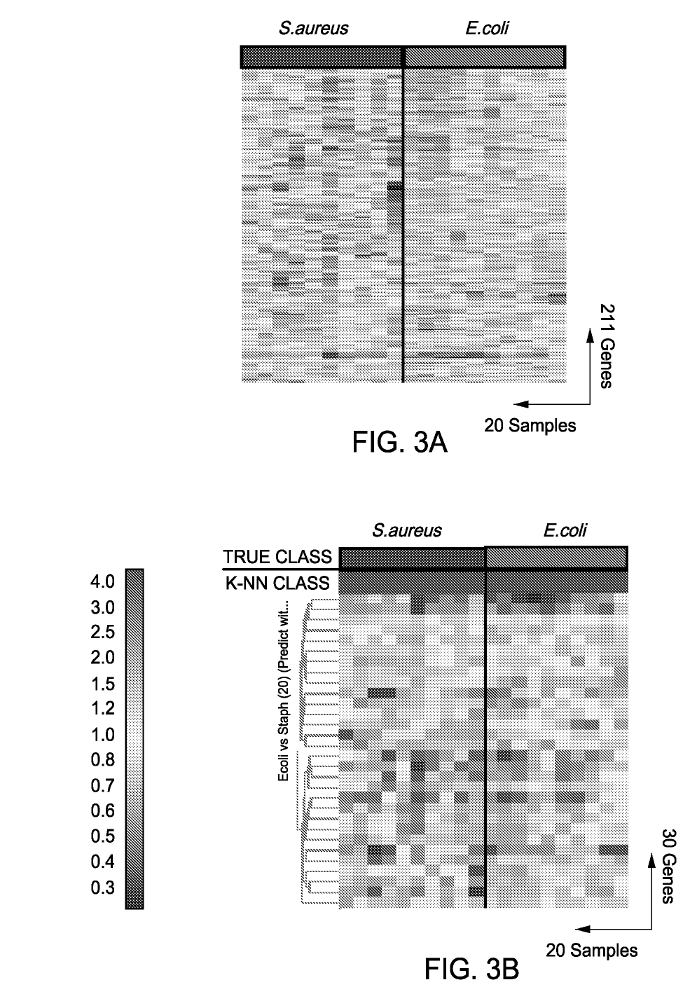

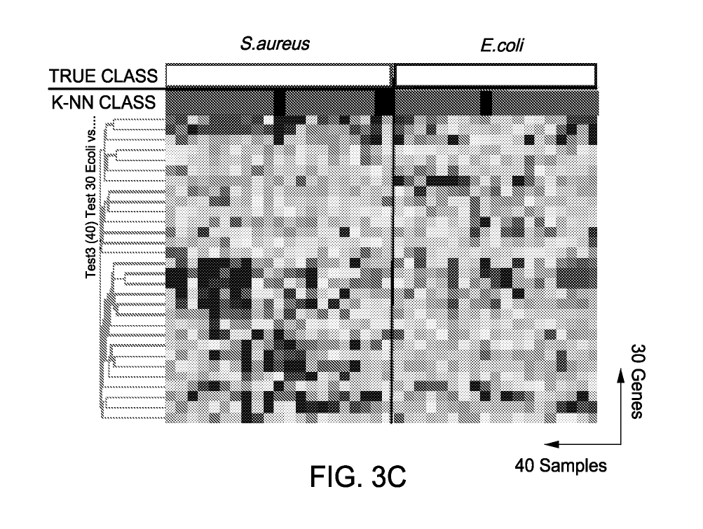

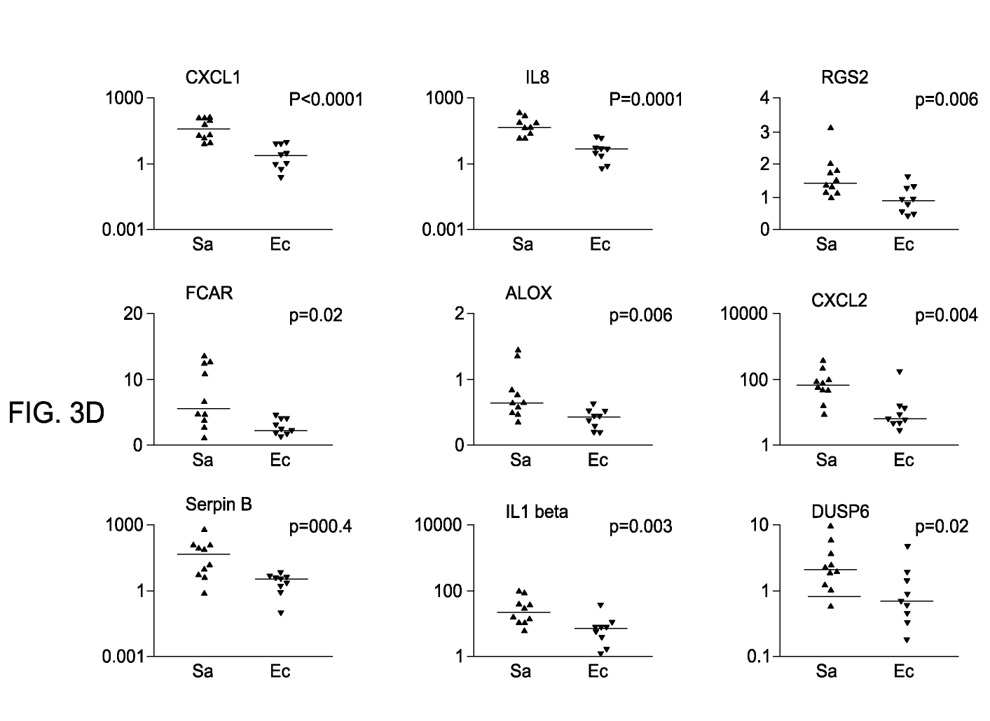

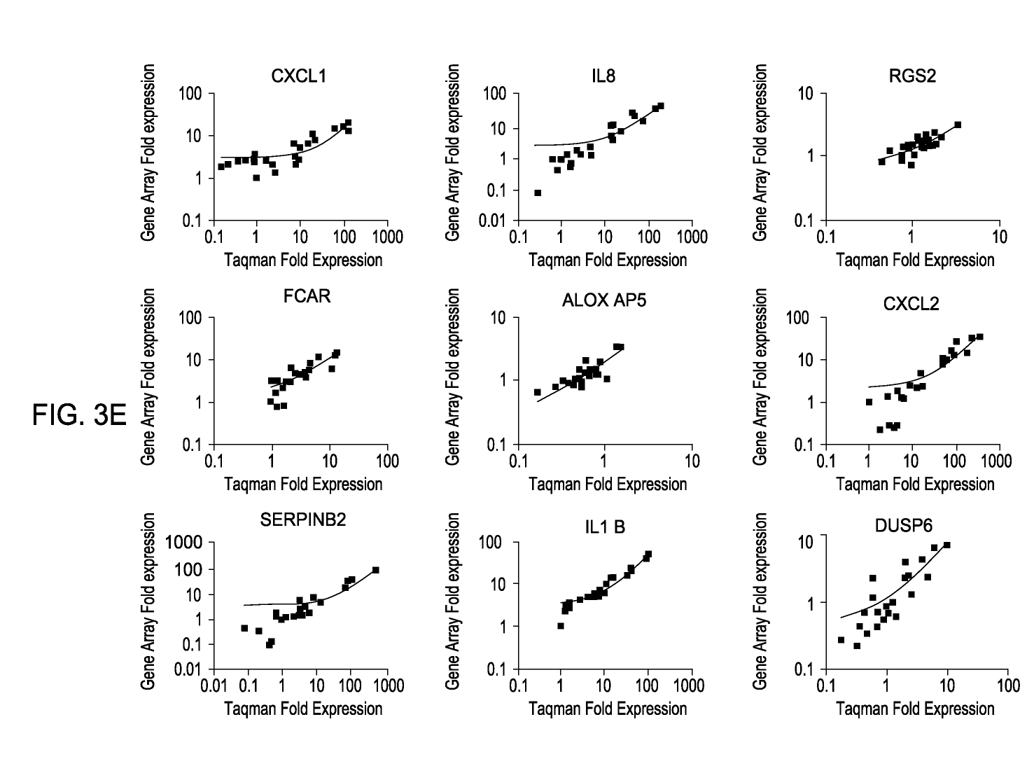

Statistical group comparisons yielded 211 genes with significantly different expression levels (p<0.01); (Supplementary Table 4 and FIG. 3a). Expression levels of a selection of genes were independently confirmed by real time PCR (FIGS. 3d and 3e). A number of genes over-expressed in S. aureus compared to E. coli are associated with neutrophil activity, including chemoattractant molecules such as CXCL1 (CXC chemokine ligand 1, GRO-1) and PPIB (cyclophilin B) [13, 14]. Furthermore, the matrix metalloproteinase 9 (MMP9) plays an important role in neutrophil extravasation and migration [15]; PRG1 (secretory granule proteoglycan 1) participates in packaging of granule proteins in human neutrophils [16]; and ALOX5AP activates arachidonate 5-lipoxygenase and prolongs the capacity of neutrophils to synthesize leukotrienes [17]. Finally, neutrophils have recently been identified as the main source of S100A8 and S100A9 (Calgranulin A and B, alias MRP 8 and 14) in a S. aureus infection model [18]. These results suggest that neutrophil activity may, in part, explain differences in levels of gene expression between samples obtained from patients with E. coli and S. aureus infections. Previous studies in patients with Systemic Lupus Erythematosus (SLE) a similar signature was traced down to the presence of low-density immature neutrophils that co-purified with mononuclear cells during density gradient centrifugation [9]. Interestingly, this “granulopoeisis signature”, which corresponds to a faster neutrophil turnover rate, can also be observed in expression profiles derived from whole blood in patients with SLE (unpublished observation).

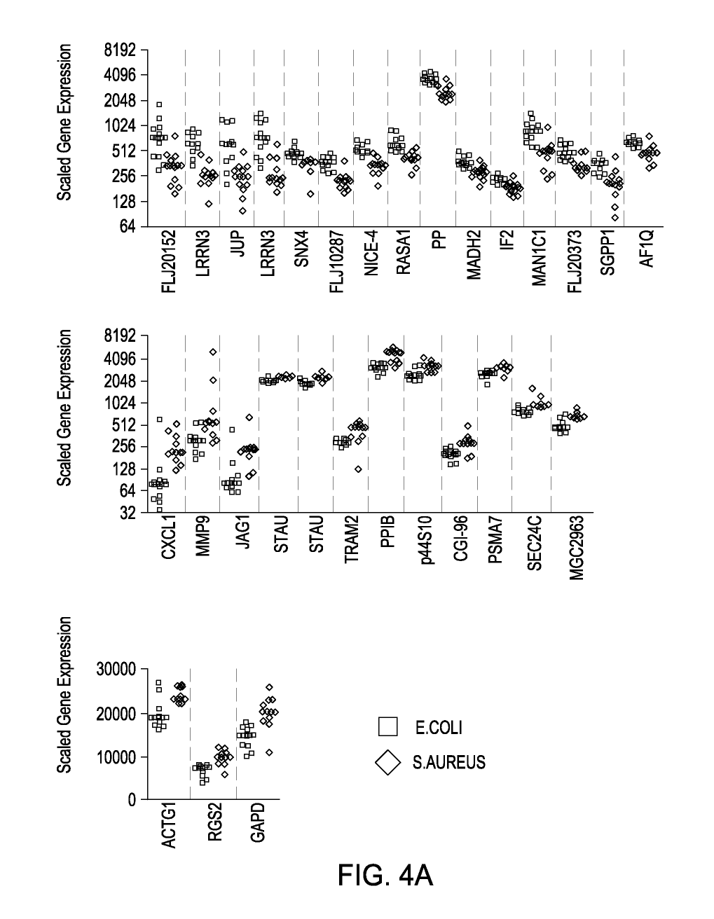

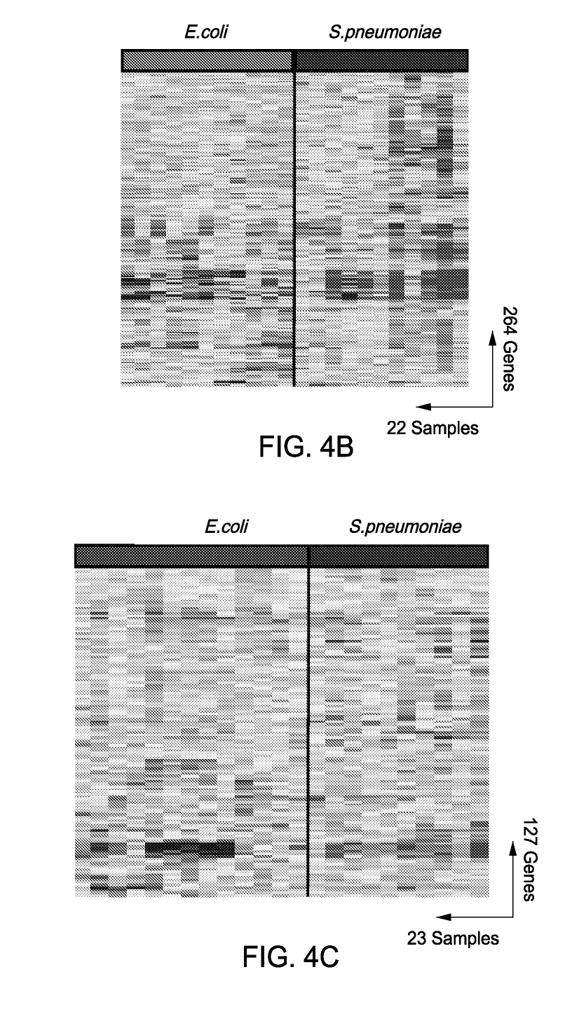

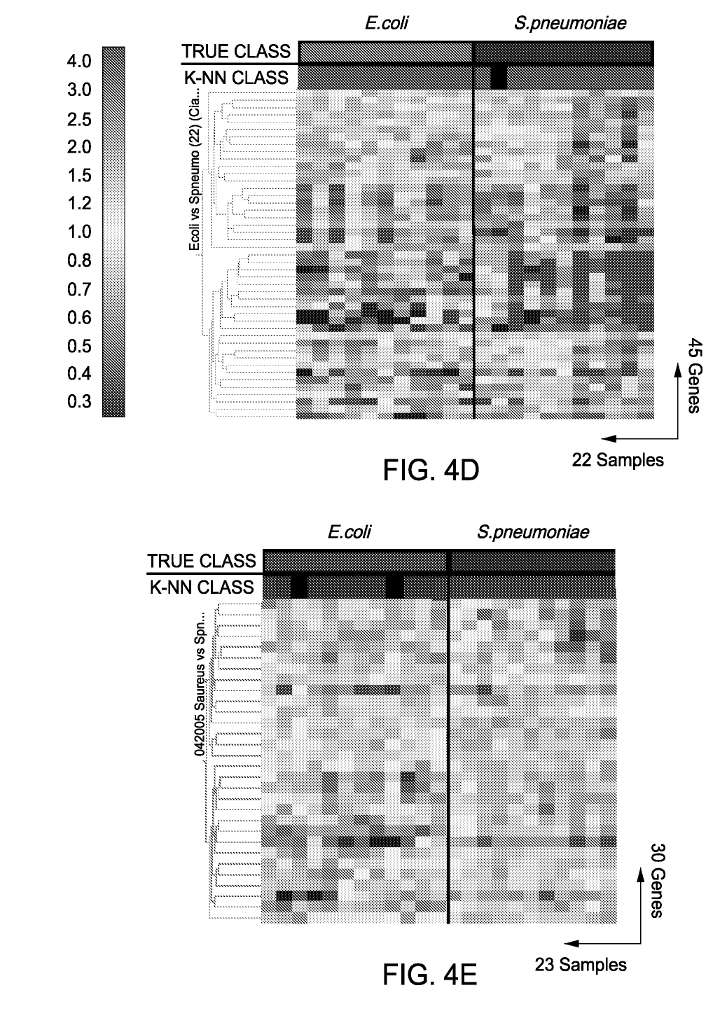

Thirty classifier genes which discriminate between the training set of patients with E. coli and S. aureus infections were identified (FIG. 4 and Table 3 and Supplementary Table 6). In leave-one-out cross-validation 19 of 20 samples were classified correctly (95% accuracy) (see also FIG. 3b). One patient with a S. aureus infection (INF 89) was misclassified. The classifier genes were validated with an independent set of patients with S. aureus (n=21) and E. coli (n=19) infections, which were again selected without regard to age or type of antibiotic treatment (S. aureus: 9 years [10 months-18 years]; E. coli: 2 months [2 weeks-5 months]). The 30 genes correctly classified 34 of the 40 samples (85% accuracy; FIG. 3c). Two samples (INF175 and INF206) were misclassified and 4 samples were indeterminate in their classification (INF168, INF220, INF281 and INF315). The greater heterogeneity of clinical disease and severity represented by the patients with S. aureus infections may contribute to the lower predictive accuracy for this group, although no specific pattern of misclassification was evident.

Thus, these results demonstrate that blood leukocyte transcriptional signatures distinguish disease etiology in patients with acute infections caused by S. aureus or by E. coli. Furthermore, notable functional convergence among discriminatory signatures were identified: Interferon-inducible genes were found among genes over-expressed in patient with Influenza A, while genes associated with neutrophils were expressed at higher level in S. aureus compared to E. coli groups.

Classifier genes discriminating samples from patients with acute influenza A, E. coli, S. aureus or S. pneumoniae infections show minimal overlap. The present inventors have now defined sets of classifier genes that discriminate patients with influenza A versus bacterial infections, and patients with E. coli versus S. aureus infections. To complete the panel of classifier genes additional pair-wise comparisons and identified sets of genes discriminating patients with S. pneumoniae infections were performed. Comparison of E. coli (n=11) and S. pneumoniae (n=11) infection groups yielded 264 significantly differentially-expressed genes (P<0.01), and 45 classifier genes (FIGS. 4b and 4c and Supplementary Tables 7 and 8); Sample class was assigned correctly for 20 of 22 samples (91% accuracy) in leave-one-out cross-validation of the training set. Comparison of S. aureus (n=12) and S. pneumoniae (n=11) infection groups yielded 127 differentially expressed genes (P<0.01) and 34 classifier genes. FIGS. 4d and 4e and Supplementary Tables 9 & 10). Sample class was assigned correctly for 19 of 23 samples (83% accuracy) in leave-one-out cross-validation of the training set.

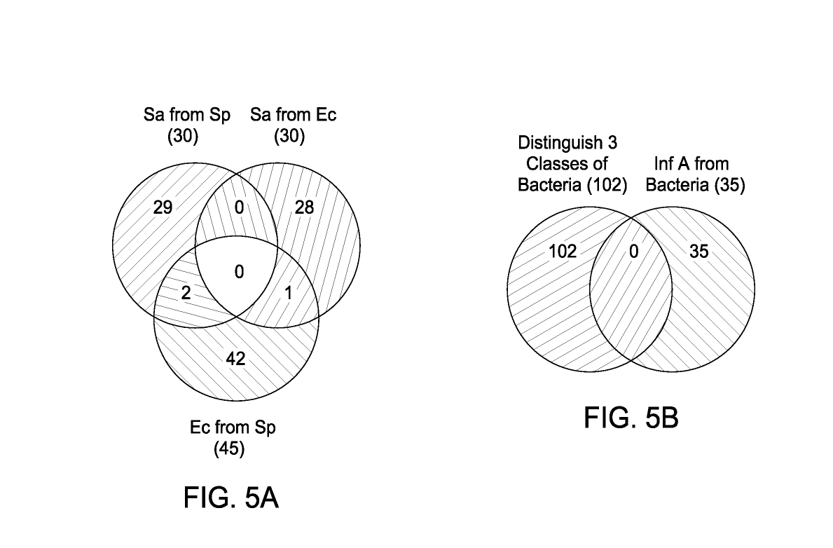

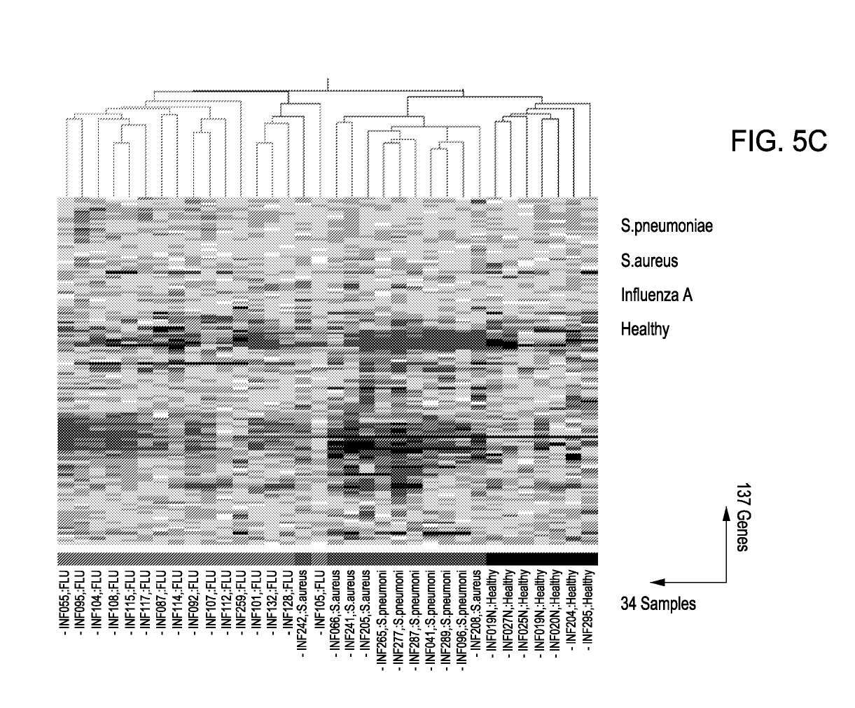

Sets of classifier genes obtained for each pair-wise analysis were systematically compared and found to be almost mutually exclusive (FIG. 5a). Furthermore, none of the 102 genes that discriminated one bacterial species from the other was necessary to distinguish influenza A from bacterial infections (FIG. 5b). Thus, multiple infectious disease etiologies can be distinguished using independent sets of transcriptional signatures.

Distinct expression patterns in patients with acute respiratory infections caused by different pathogens. Gene expression patterns in a mixed cohort of patients presenting with the same clinical manifestations were examined. Sets of classifier genes identified throughout this study (FIGS. 5a and 5b) were merged, and used to generate expression patterns for a subset of patients with lower respiratory tract infections (27 samples listed Table 1). Seven samples collected from healthy volunteers were used as a reference (Table 1). Hierarchical clustering of genes and samples identified four prototypical expression signatures: Healthy controls were clearly distinguishable from all the infectious disease groups based on PBMC expression profiles. This finding is in itself remarkable, since none of the training sets used to generate the classifiers included samples from healthy volunteers. A second signature was associated with samples from patients with influenza A infection (including interferon-inducible genes) and was clearly different from a third signature, which characterized infections caused by S. aureus and S. pneumoniae (including neutrophil-associated genes). Distinctions between these two gram positive bacteria were minimized by the overt dominance of signatures differentiating the three major classes of samples.

Interestingly, four samples belonging to the influenza A group and one from the S. aureus group were characterized by a fourth signature, which combined elements of the previous ones (interferon-inducible and neutrophil-associated genes: FIG. 5c, indicated by the asterisk). This finding suggests one of at least two possibilities: 1) the mixed signatures arise as the result of co-infections that could not be detected by routine diagnostic methods, or 2) the analysis of PBMC transcriptional signatures can reveal the existence of distinct patient subgroups. A larger patient cohort will be necessary to investigate these possibilities and identify potential clinical implications. Further review of the medical records of the 5 patients with mixed signature, identified 3 patients with influenza (#101, #128 and #132) who had radiological evidence of pneumonia and white blood cell differential counts with 11%, 16% and 28% bands, respectively. The evidence suggests the possibility of co-infections in these 3 cases. These results clearly demonstrate that discriminative blood leukocyte transcriptional patterns can be obtained in patients presenting similar symptoms.

Results can be reproduced in a novel independent set of samples and across microarray platforms. The study design includes a training set for the identification of classifiers (FIG. 1b; influenza vs. Bacteria; n=23 samples) and a test set to validate independently these findings (FIG. 1c Influenza vs. bacteria n=37 samples; and FIG. 1d an additional 31 patients infected with S. aureus). These data, obtained from a total of 91 patients, were generated using Affymetrix U133A and U133B GeneChips. Data validation was taken one step further in order to further confirm these findings, and carried out a similar analysis on additional sets of patients using different microarray platforms. A new cohort of 22 patients was recruited with acute influenza/bacterial infection and analyzed PBMC samples using the most recent version of Affymetrix GeneChips (U133 plus 2.0).

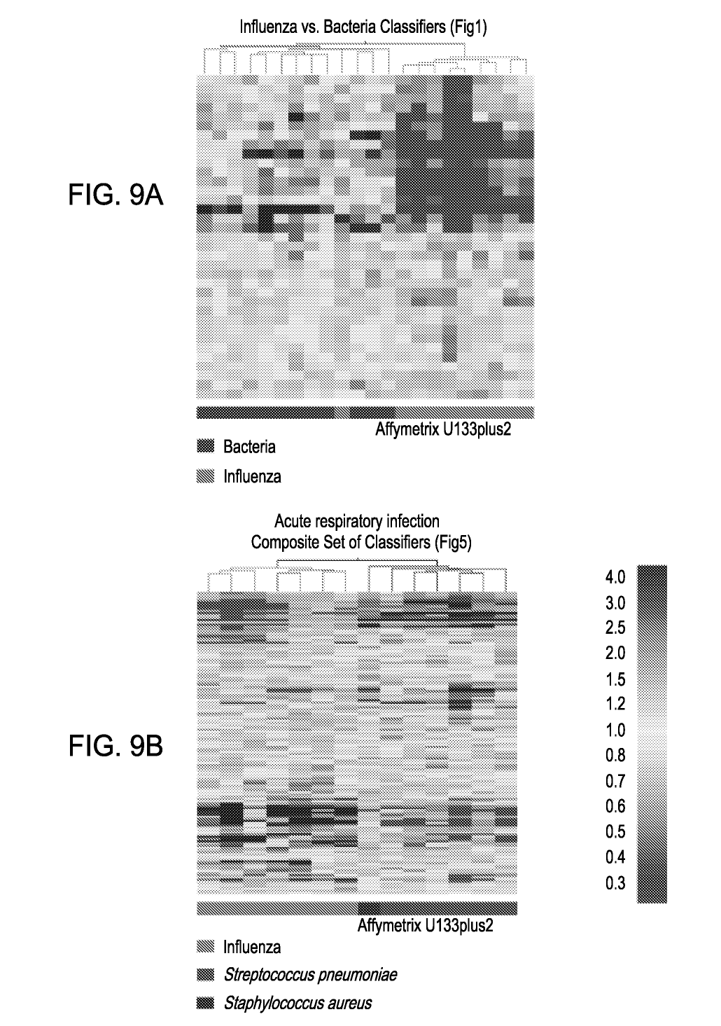

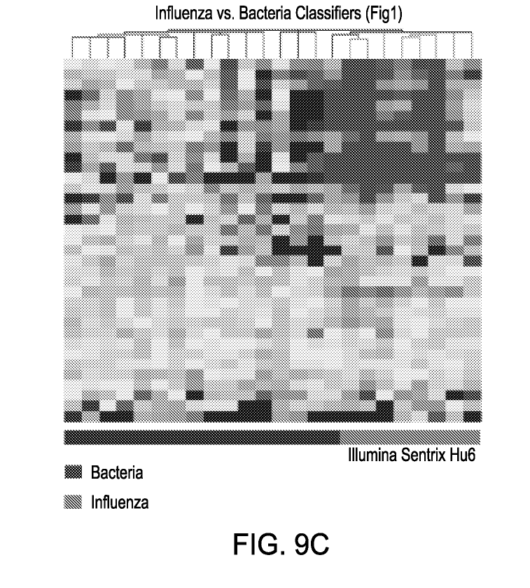

FIGS. 9a to 9c summarize independent confirmation and validation across microarray platforms. FIG. 9a shows the results from a new set of patients with acute influenza (n=10) or bacterial infection (S. aureus; n=6; S. pneumoniae: n=6) analyzed using Affymetrix U133 plus 2.0 GeneChips. Classifier genes used to discriminate influenza A from bacterial infections (35 genes, Venn diagram, right; FIG. 1 and Supplementary Table 3) were used to cluster this new set of samples. In FIG. 9b, a subset of 14 samples from patients with acute respiratory infection included in FIG. 9a were clustered using the list of 137 transcripts from FIG. 5. FIG. 9c shows the results from another independent set of samples (none of which being used in any of the previous analyses) was obtained from a new set of patients with acute influenza (n=8) or bacterial infection (S. aureus; n=13; S. pneumoniae: n=3) analyzed using Illumina Sentrix Hu6 whole genome BeadChips. Classifier genes used to discriminate influenza A from bacterial infections (35 genes, Venn diagram, right; FIG. 1 and Supplementary Table 3) were used to cluster this new set of samples. Transformed expression levels are indicated by color scale, with red representing relative high expression and blue indicating relative low expression compared to the median expression for each gene across all donors.

The present invention was able to distinguish almost perfectly infections caused by S. aureus or S. pneumoniae from infections caused by influenza (FIG. 9a; one influenza sample grouped in the bacterial infection cluster), and to obtain discriminative signature in patients with acute respiratory infection (FIG. 9b). Microarray data are notoriously difficult to compare across totally different platforms [19, 24-26], but the present invention was able to, once again, reproduce the initial results when analyzing a new set of 24 samples using Illumina's whole genome Sentrix Hu6 BeadChips (FIG. 9c; one sample from the bacterial infection group clustered with influenza samples). In this cohort, only two patient belonging to the S. aureus or S. pneumoniae group presented with acute respiratory infection.

As such, 148 microarray analyses were conducted, including 141 on samples collected from patients with acute infections. Along with the confirmation obtained by real-time PCR (FIG. 3d) the independent data validation carried out across microarray platforms attest to the robustness of these findings.

Distinct transcriptional signatures differentiate patients with acute infection from those with autoimmune disease. Interferon-inducible genes were found to be over-expressed in patients with acute influenza infection. An interferon signature was also identified previously in blood leukocytes of patients with Systemic Lupus Erythematosus [9]. Next, whether gene expression patterns in blood leukocytes would nevertheless permit differentiation of influenza infection from SLE was determined. Samples obtained from SLE patients were compared to their respective healthy control group. Similarly, patients from the various infectious disease groups were compared to an appropriate cohort of healthy volunteers (11 patients per group: influenza A, E. coli, S. aureus, S. pneumoniae, compared to 9 healthy controls). P-values obtained for each comparison (overall, 5 sets of patients versus their respective control groups) were compiled and collectively analyzed. This approach recapitulates changes observed across multiple studies and a large number of samples, and is particularly well suited when all potentially confounding factors cannot be accounted for (e.g., SLE incidence is much higher in females). Significance patterns were analyzed in order to evaluate the overlap between the gene expression signatures obtained for the influenza and SLE groups (FIG. 6). Filtering criteria were applied to select transcripts over- or under-expressed in both groups of patients in comparison to their respective control group (FIG. 6, upper panel). It was found that among over-expressed transcripts a cluster including interferon-inducible genes (FIG. 6: upper panel—IFN; Supplementary Table 11), that were significantly changed in both influenza and SLE groups, but not in patients with bacterial infections. Conversely, genes that changed significantly versus healthy controls in one group (FLU or SLE, p<0.01), but not the other (p>0.5; FIG. 6, middle and lower panels) could also be identified. This approach revealed disease-specific signatures (data not shown). Several clusters uniquely characterizing influenza A patients can be found in FIG. 6 and Supplementary Tables 1 to 11). These results further demonstrate that perturbations of blood leukocyte transcriptional profiles are disease-specific.

The comparative analysis of a compendium of host-pathogen microarray datasets (encompassing 32 studies) identified both common host transcriptional responses to infections and as pathogen-specific signatures [27]. Broad similarities exist, with for instance dynamic cascades of cytokines and chemokines involved in the activation and recruitment of immune cells being observed in the context of fungal, bacterial or viral infections 30-34. However, two factors contribute to the specificity of transcriptional responses to infections: 1) the diversity of the molecular mechanisms involved in pathogen recognition; and 2) alterations of host responses by pathogens. Upon activation, Toll-like receptor (TLR) family members trigger signaling pathways that share common components while retaining unique characteristics accounting for the specificity of transcriptional responses. Hence, qualitative and quantitative differences in the responses to gram-positive and gram-negative bacteria, respectively recognized by TLR2 and TLR4, have been observed. Furthermore, responses measured in dendritic cells exposed to influenza virus (through TLR3), E. coli (through TLR4), and Candida (through TLR2/TLR4) were also found to be markedly different. Reprogramming of host cells by pathogens also contributes significantly to the diversification of transcriptional responses to infection. As measured by microarrays mycobacterial products are for instance able to inhibit interferon gamma induced gene regulation in macrophages [28]. Similarly, microarray studies have demonstrated the ability of herpes virus, pseudorabies virus, hepatitis C, varicella-zoster virus or rhinovirus to limit the ability of the host to develop effective anti-viral responses by a variety of mechanisms. Altogether the vast body of experimental data accumulated over the past years suggests that hosts can mount pathogen-specific transcriptional responses to infections.

A number of studies have shown that different transcriptional programs could be triggered upon exposure of immune cells to various pathogens in vitro [19-22]. Here, it was demonstrated that gene expression patterns in blood leukocytes can be used to distinguish acute infections caused by four different pathogens: influenza A virus; the Gram negative bacterium, E. coli; and Gram-positive bacteria S. aureus and S. pneumoniae, which are among the most common infections leading to child hospitalization.

Two parameters might account for differences in gene expression levels observed in blood leukocytes: 1) changes in transcriptional activity (e.g., up-regulation of interferon-inducible genes) and/or 2) an altered cellular composition of blood samples (e.g., neutrophil signature). Changes in expression due to either one or both of these parameters may be mediated directly by pathogen-derived molecules or the action of secondary factors released by the host (e.g., cytokines). Major differences were observed in the cellular composition of blood samples obtained from the different groups of patients. Indeed, it is well established in clinical practice that the routine white blood cell and differential counts can not distinguish between viral and bacterial infections and much less between infections caused by gram positive and gram negative bacteria. However, the present inventors have found that subtle differences might account for observed transcriptional signatures as exemplified by the neutrophils signature in Systemic Lupus Erythematosus which is due to enhanced efflux of low density neutrophils present in PBMC preparations. The site of disease involvement may also influence expression profiles observed in blood leukocytes and reflects the predilection of certain species of pathogens for different infection sites. E. coli, for example, is more likely to cause urinary tract infection, while the most common clinical manifestations of S. aureus are skin/soft tissue infections and osteomyelitis. The results obtained in the present study suggest, however, that distinctive expression signatures can be found in the context of a single disease manifestation. Indeed, when analyzing samples from patients with lower respiratory infections a clear separation between infections caused by the different pathogens was observed, confirming the existence of pathogen-associated transcriptional signatures.

The ability to identify etiologic agents responsible for acute infections remains disappointingly low in many clinical situations, and the analysis of blood leukocyte transcriptional profiles has the potential to transform the diagnosis of infectious diseases. In contrast to microbial cultures, serologic assays or even PCR-based tests, leukocyte gene expression results can be obtained quickly and reliably regardless of the site of disease involvement. This information should allow prompt initiation of adequate anti-infective therapy and establishment of the appropriate infection control measures. Furthermore, transcriptional analysis of blood leukocytes provides information about the patient that can be used for disease diagnosis and potentially as markers of disease progression and prognosis. Compared to the inflammatory markers such as white blood cell counts, erythro-sedimentation rate and C-reactive protein, which have traditionally been used as indicators of disease evolution, gene expression arrays provide comprehensive molecular picture that not only reflects the relative cellular composition of the tissue but also gene regulation resulting from ongoing immune reactions and/or pathogen exposure.

These results demonstrate the value of transcriptional signature analysis in blood leukocytes as an adjunctive method of diagnosis of infectious diseases for both single, on the spot analysis, but also for the detection, determination, evaluation, prognosis, diagnosis and prediction of infectious disease outcome, acute, chronic or both. Large multi-center studies will be necessary to collect and independently evaluate large numbers of samples, eventually bringing blood leukocytes gene expression profiling closer to a routine clinical diagnostic application.

Patient Information. Blood samples were obtained from 29 patients with E. coli infections (median age: 2 months; range: 2 weeks-16 years), 31 patients with S. aureus infections (7 years; 3 months-18 years), 13 with S. pneumoniae (2 years; 2 months-16 years), 18 with influenza A infections (1.5 years; 3 weeks-36 years), and 7 healthy controls (11 months; 3 months-22 months). Patients were divided into training and test sets according to age and antibiotic treatment (Table 1). All subjects with acute infections and their controls were recruited at Children's Medical Center Dallas (CMC), while the SLE patients and their respective controls were recruited at Texas Scottish Rite Hospital. The study was approved by the Institutional Review Boards and informed consent was obtained for all patients. Microbiologic diagnosis was established by standard bacterial cultures of relevant tissue specimens or blood, and by direct fluorescent antigen testing and viral cultures. All potentially eligible patients were identified on a daily basis by the investigators from both the microbiology laboratory database and inpatient admissions records. A second step was then undertaken to confirm eligibility on the basis of history, clinical findings, bacterial and viral cultures, and immunofluorescence tests. Patients with suspected (by clinical findings) or documented (by microbiologic tests) polymicrobial infections, history of immunodeficiency, chronic disease or receiving steroids or other immunomodulatory agents, were excluded. Patients were enrolled once a confirmed microbiologic diagnosis was established. Systematic testing for the presence of concomitant viral infection was initiated after the beginning of the study and respiratory viral cultures were performed in 60 of 73 (82%) patients with bacterial infections. Control samples were obtained from healthy individuals scheduled to undergo elective surgical procedures, and from healthy outpatient clinic patients.

Processing of Blood Samples. All blood samples were collected in acid citrate dextrose tubes (BD Vacutainer) at Children's Medical Center or Texas Scottish Rite Hospital, Dallas, Tex. and immediately delivered at room temperature to the Baylor Institute for Immunology Research, Dallas, Tex., for processing. Peripheral blood mononuclear cells (PBMCs) from 3-4 ml of blood were isolated via Ficoll gradient and immediately lysed in RLT reagent (Qiagen, Valencia, Calif.) with beta-mercaptoethanol (BME) and stored at −80° C. (within 4-6 hours from the time of blood draw) in the same laboratory by the same team to standardize the quality and handling of RNA samples.

Microarray assay. Total RNA was isolated using the RNeasy kit (Qiagen, Valencia, Calif.) according to the manufacturer's instructions and RNA integrity was assessed by using an Agilent 2100 Bioanalyzer (Agilent, Palo Alto, Calif.). Double-stranded cDNA was generated from 2-5 micrograms of total RNA, followed by single-round in vitro transcription with biotin-labeled nucleotides, using the Affymetrix RNA transcript labeling kits (Affymetrix Inc, Santa Clara, Calif.). Biotinylated cRNA targets were purified using the Sample Cleanup Module (Affymetrix), and subsequently hybridized, according to the manufacturer's standard protocols, to Affymetrix HGU133A GeneChips (which contain 22,283 probe sets). Arrays were scanned using an Affymetrix confocal laser scanner. Expression results of a set of genes were confirmed by real time PCR.

Real-time RT-PCR analysis. Total RNAs were subjected to a second DNase treatment with the TURBO DNA-free kit (Ambion Inc., Austin, Tex.). cDNA was synthesized using the Two-Cycle cDNA Synthesis kit (Affymetrix) followed by in vitro transcription (MEGAscript T7 kit, Ambion, Inc., Austin, Tex.). Two-step RT-PCR was performed using Applied Biosystems TaqMan Assays on Demand probe and primer sets according to the manufacturer's instructions. Reverse 6 transcription was carried out using the High Capacity cDNA Archive Kit (Applied Biosystems). Real-time PCR was performed on an ABI Prism 7700 Sequence Detection System. Human β-glucuronidase (GUSB) was chosen from a panel of 10 human endogenous controls as the most constitutively expressed in the samples and was therefore used as the reference gene for normalization. Relative mRNA expression was calculated using the comparative cycle time (CT) method according to the manufacturer's instructions. Results were calculated as the normalized difference in CT for a given patient with infection relative to one healthy donor as baseline whose expression is closest to the mean of all healthy donors (AACT).

Illumina BeadChips: These microarrays consist of 50mer oligonucleotide probes attached to 3 μm beads, which are lodged into microwells at the surface of a glass slide. Samples were processed and data acquired by Illumina Inc. (San Diego, Calif.). Targets were prepared using the Illumina RNA amplification kit (Ambion, Austin, Tex.). cRNA targets were hybridized to Sentrix Hu6 BeadChips (>46,000 probes), which were scanned on an Illumina BeadStation 500. Illumina's Beadstudio software was used to assess fluorescent hybridization signals.

Raw data obtained for all 148 samples analyzed are deposited with GEO (www.ncbi.nlm.nih.gov/geo/) (accession number ______).

Microarray data analysis. Microarray Suite, Version 5.0 (MAS 5.0; Affymetrix) software was used to assess fluorescent hybridization signals, to normalize signals, and to evaluate signal detection calls. Raw signal intensity values for each probe set were analyzed by algorithms in MAS 5.0. A maximum of eight samples were assigned randomly for hybridization and staining each run day in order to minimize technical variability.

Normalization of signal values per chip was achieved using the MAS 5.0 global method of scaling to the target intensity value of 500 per GeneChip. Analysis was restricted to probe sets for which a P (present) call was obtained in at least 75% of GeneChips in at least one patient class evaluated (quality control probes). A gene expression analysis software program, GeneSpring, Version 7.1 (Agilent), was used to perform statistical analysis, hierarchical clustering and classification of samples. Nonparametric univariate tests (Mann-Whitney U or Fishers exact test) were used to rank genes on the basis of their ability to discriminate between pre-defined groups of patients. The ability of the top ranked (i.e., classifier) genes to discriminate the pre-defined class of pathogen was determined by the K-Nearest Neighbors (kNN) method [23].

K-Nearest Neighbors (kNN) method: (1) The algorithm ranks the genes by their predictive strengths, the negative natural log of the p-value as determined by nonparametric tests; and (2) Leave-one-out cross validation was used to estimate the prediction error rate (or accuracy) by the systematic-removal of one donor from the known samples to use as a test sample. This process is repeated until all the donors have been “tested.” The discriminating gene lists from both Mann-Whitney U and Fisher's exact test were combined and used for discrimination between sample classes. (3) To assign sample class, the algorithm evaluates class by testing the number of known classes nearest to the sample of unknown class, based on Euclidean distance of normalized expression intensity, and computes a p-value. The class with the lowest p-value is assigned to the unknown sample. A p-value ratio cut-off of 0.5 was used in all discrimination analyses. A class will be assigned to a sample, if the p-value from the predicted class is at least 2 times less than the other class (e.g., p-value of influenza A class/p-value of bacteria class).

Analysis of significance patterns. Statistical comparisons between each group of patients and its respective healthy control group were performed (Mann-Whitney rank test). Genes significantly changed (p<0.01) were divided into two sets: over-expressed versus control and under-expressed versus control for the two reference groups (FLU and SLE). Sets of genes were identified by applying selection criteria to these group(s) (e.g. P<0.01 in FLU and P>0.5 in SLE). P-values for these genes were obtained in the context of other diseases (comparison groups: S. aureus, S. pneumoniae, E. coli). P-values for comparison groups were set to 1 when changes in gene expression were opposite from that of the reference group. P-value data were processed with GeneSpring, Version 7.2 (Agilent), which was used to perform hierarchical clustering and group genes based on significance patterns.

Transcriptional signatures discriminate patients with influenza A infection from those with bacterial infection. Using a standard gene-level analysis it was found that microarray analysis can be used to differentiate viral infections (influenza A) from bacterial infections (E. coli and S. pneumoniae) as illustrated in FIG. 1C. FIG. 1C shows the gene expression signatures discriminate influenza A from bacterial infections. Thirty-five classifier genes that best discriminate patients with influenza A virus infection from patients with bacterial infections (E. coli or S. aureus) with 91% (21/23) accuracy in training set were then independently validated in a test set (n=37). These 35 predictors classified the test set with 95% accuracy (35/37).

Module-level microarray data analysis. This strategy is based on the initial extraction of 28 sets of coordinately expressed genes (regrouping nearly 5000 transcripts), or transcriptional modules, from a large microarray gene expression dataset (8 diseases, nearly 250 samples×44,000 transcripts). These modules were subsequently used as building blocks for analyses that were carried out on a module-by-module basis: functional interpretation through literature analysis first, then group comparison between samples obtained from healthy subjects and patients with acute infections.

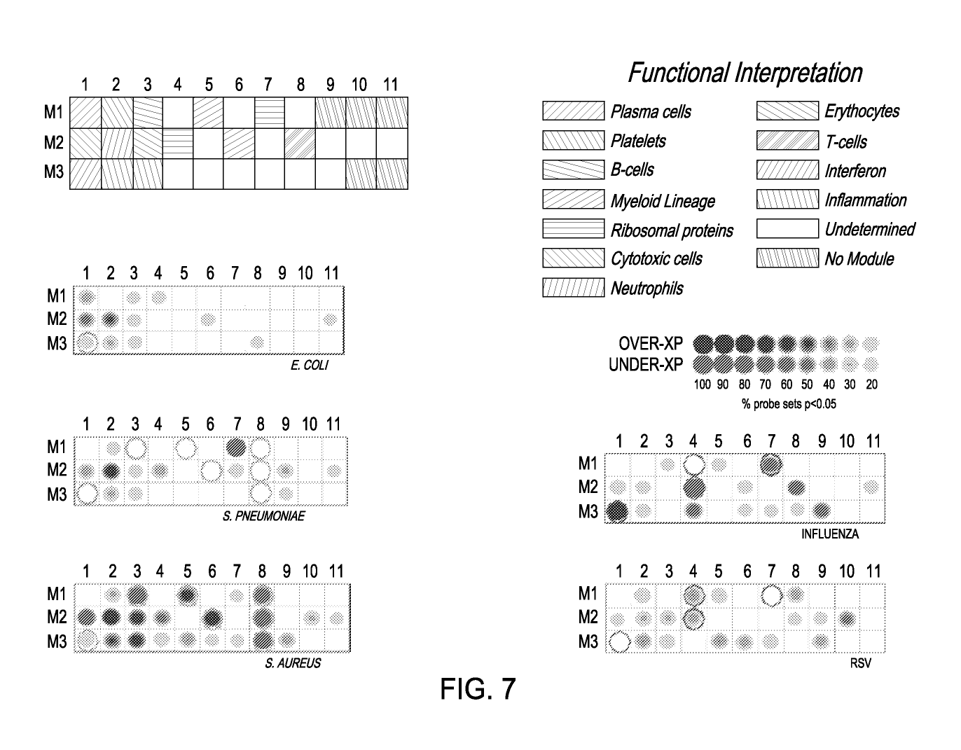

FIG. 7 shows gene vectors that may be used for mapping transcriptional changes at the module-levels identifies disease-specific patterns. Group comparisons were carried out between patients and uninfected individuals on a module-by-module basis. The spots represent the percentage of significantly over-expressed (red) or under-expressed (blue) genes within a module. This information is displayed on a grid with the coordinates corresponding to one of 28 module IDs (e.g. Module M3.1 is at the intersection of the third row & first column).

The gene vector and mapping approach permits reducing noise levels and facilitates data interpretation. The group at Dallas has also demonstrated that modular transcriptional data were reproducible across microarray platforms.

Identification of diagnostic markers in the blood of patients. Mapping global transcriptional changes at the module-level has helped with the interpretation of patient PBMC transcriptional profiles. It also revealed disease-specific combinations of modular changes. This is illustrated in FIG. 7, comparing changes in module M3.1 (interferon, circled in green—the proportion of differentially over- or under-expressed transcripts indicated by a red and blue spot, respectively) across infections caused by gram positive and negative bacteria. Differences were also found between the two species of gram positive bacteria (FIG. 7, orange circles). Most interestingly, marked differences were observed in the modular profiles of influenza and RSV, despite the fact that these viral infections have similar clinical presentations. The complete absence of induction of interferon-inducible transcripts in patients with RSV was a striking difference from influenza infection which was associated with a powerful interferon response in patients (M3.1). Other differences were observed, most strikingly with modules M1.4 and M1.7 (highlighted in purple in FIG. 7).

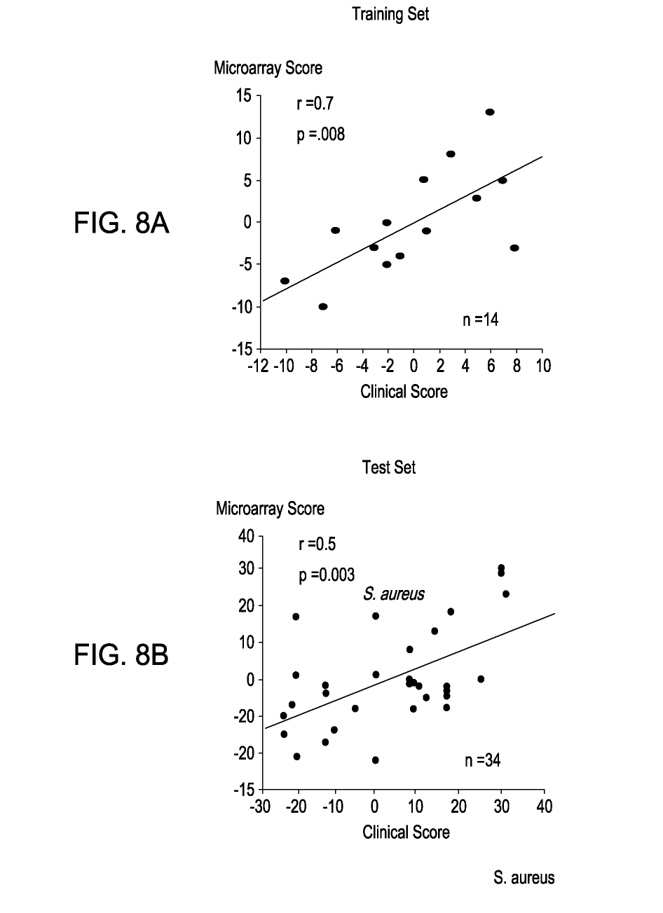

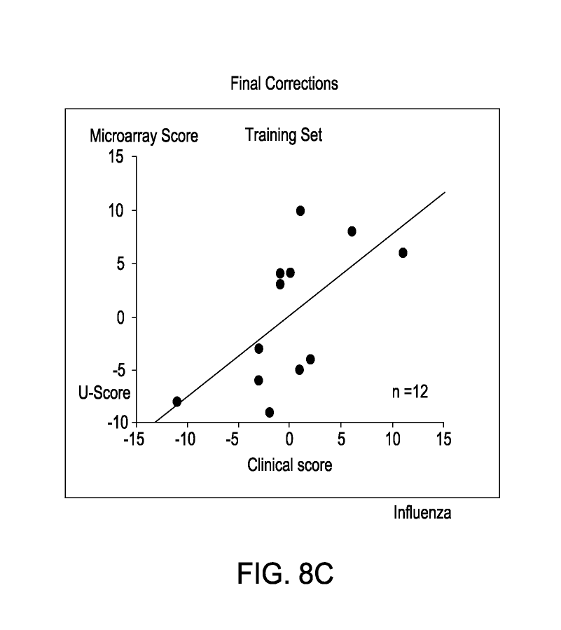

Identification of markers of disease severity: The tools currently available for the diagnosis of infectious diseases rely on the direct detection of the pathogen (e.g. by culture, staining or PCR). In comparison with these methods monitoring gene expression changes of the patient's immune cells offers the possibility of predicting the severity of the disease. Indeed, modular expression levels correlated (averaged values across transcripts) with clinical indicators of disease severity. The modules correlating (positively or negatively) with severity were then consolidated in a single score after carrying out multivariate analysis based on U-statistics (generating “U-scores”—results for S. aureus and influenza are shown in FIG. 8).

FIG. 8 shows the microarray scores for the assessment of disease severity in patients with acute infections. Module-level microarray expression data were combined in a single score through a multivariate analysis based on U-statistics. The microarray scores thus obtained were correlated with a clinical score constituted by relevant indicators of disease severity (e.g. fever, hypotension, acidiosis). Markers were identified in a training set and validated in an independent set of patients (test set). Thus a unique microarray-based blood assay produces clinical information that can be used: (1) to determine disease etiology; and (2) to assess disease severity in patients with acute infections. FIGS. 9a to 9c summarize independent confirmation and validation across microarray platforms.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

1. Fauci, A. S. 2005. The global challenge of infectious diseases: the evolving role of the National Institutes of Health in basic and clinical research. Nat Immunol 6:743-747.
2. Relman, D. A. 2002. New technologies, human-microbe interactions, and the search for previously unrecognized pathogens. J Infect Dis 186 Suppl 2:S254-258.
3. Fauci, A. S. 2004. Emerging infectious diseases: a clear and present danger to humanity. Jama 292:1887-1888.
4. Medzhitov, R., and C. A. Janeway, Jr. 1997. Innate immunity: the virtues of a nonclonal system of recognition. Cell 91:295-298.
5. Medzhitov, R., and C. Janeway, Jr. 2000. Innate immune recognition: mechanisms and pathways [In Process Citation]. Immunol Rev 173:89-97.
6. Alizadeh, A. A., M. B. Eisen, R. E. Davis, C. Ma, I. S. Lossos, A. Rosenwald, J. C. Boldrick, H. Sabet, T. Tran, X. Yu, J. I. Powell, L. Yang, G. E. Marti, T. Moore, J. Hudson, Jr., L. Lu, D. B. Lewis, R. Tibshirani, G. Sherlock, W. C. Chan, T. C. Greiner, D. D. Weisenburger, J. O. Armitage, R. Warnke, R. Levy, W. Wilson, M. R. Grever, J. C. Byrd, D. Botstein, P. O. Brown, and L. M. Staudt. 2000. Distinct types of diffuse large B-cell lymphoma identified by gene expression profiling. Nature 403:503-511.
7. Golub, T. R., D. K. Slonim, P. Tamayo, C. Huard, M. Gaasenbeek, J. P. Mesirov, H. Coller, M. L. Loh, J. R. Downing, M. A. Caligiuri, C. D. Bloomfield, and E. S. Lander. 1999. Molecular classification of cancer: class discovery and class prediction by gene expression monitoring. Science 286:531-537.
8. van de Vijver, M. J., Y. D. He, L. J. van't Veer, H. Dai, A. A. Hart, D. W. Voskuil, G. J. Schreiber, J. L. Peterse, C. Roberts, M. J. Marton, M. Parrish, D. Atsma, A. Witteveen, A. Glas, L. Delahaye, T. van der Velde, H. Bartelink, S. Rodenhuis, E. T. Rutgers, S. H. Friend, and R. Bernards. 2002. A gene-expression signature as a predictor of survival in breast cancer. N Engl J Med 347:1999-2009.
9. Bennett, L., A. K. Palucka, E. Arce, V. Cantrell, J. Borvak, J. Banchereau, and V. Pascual. 2003. Interferon and granulopoiesis signatures in systemic lupus erythematosus blood. Exp Med 197:711-723.
10. Rubins, K. H., L. E. Hensley, P. B. Jahrling, A. R. Whitney, T. W. Geisbert, J. W. Huggins, A. Owen, J. W. Leduc, P. O. Brown, and D. A. Relman. 2004. The host response to smallpox: analysis of the gene expression program in peripheral blood cells in a nonhuman primate model. Proc Natl Acad Sci USA 101: 15190-15195.
11. Baechler, E. C., F. M. Batliwalla, G. Karypis, P. M. Gaffney, W. A. Ortmann, K. J. Espe, K. B. Shark, W. J. Grande, K. M. Hughes, V. Kapur, P. K. Gregersen, and T. W. Behrens. 2003. Interferon-inducible gene expression signature in peripheral blood cells of patients with severe lupus. Proc Natl Acad Sci USA 100:2610-2615.
12. Hoebe, K., and B. Beutler. 2004. LPS, dsRNA and the interferon bridge to adaptive immune responses: Trif, Tram, and other TIR adaptor proteins. J Endotoxin Res 10:130-136.
13. Yurchenko, V., M. O'Connor, W. W. Dai, H. Guo, B. Toole, B. Sherry, and M. Bukrinsky. 2001. CD147 is a signaling receptor for cyclophilin B. Biochem Biophys Res Commun 288:786-788.
14. Geiser, T., B. Dewald, M. U. Ehrengruber, I. Clark-Lewis, and M. Baggiolini. 1993. The interleukin-8-related chemotactic cytokines GRO alpha, GRO beta, and GRO gamma activate human neutrophil and basophil leukocytes. J Biol Chem 268: 15419-15424.
15. Nagaoka, I., and S. Hirota. 2000. Increased expression of matrix metalloproteinase-9 in neutrophils in glycogen-induced peritoneal inflammation of guinea pigs. Inflamm Res 49:55-62.
16. Niemann, C. U., J. B. Cowland, P. Klausen, J. Askaa, J. Calafat, and N. Borregaard. 2004. Localization of serglycin in human neutrophil granulocytes and their precursors. J Leukoc Biol 76:406-415.
17. Pouliot, M., P. P. McDonald, P. Borgeat, and S. R. McColl. 1994. Granulocyte/macrophage colony-stimulating factor stimulates the expression of the 5-lipoxygenase-activating protein (FLAP) in human neutrophils. J Exp Med 179: 1225-1232.
18. Herndon, B. L., S. Abbasi, D. Bennett, and D. Bamberger. 2003. Calcium-binding proteins MRP 8 and 14 in a Staphylococcus aureus infection model: role of therapy, inflammation, and infection persistence. J Lab Clin Med 141:110-120.
19. Chaussabel, D., R. T. Semnani, M. A. McDowell, D. Sacks, A. Sher, and T. B. Nutman. 2003. Unique gene expression profiles of human macrophages and dendritic cells to phylogenetically distinct parasites. Blood 102:672-681.
20. Nau, G. J., J. F. Richmond, A. Schlesinger, E. G. Jennings, E. S. Lander, and R. A. Young. 2002. Human macrophage activation programs induced by bacterial pathogens. Proc Natl Acad Sci USA 99:1503-1508.
21. Boldrick, J. C., A. A. Alizadeh, M. Diehn, S. Dudoit, C. L. Liu, C. E. Belcher, D. Botstein, L. M. Staudt, P. O. Brown, and D. A. Relman. 2002. Stereotyped and specific gene expression programs in human innate immune responses to bacteria. Proc Natl Acad Sci USA 99:972-977.
22. Huang, Q., D. Liu, P. Majewski, L. C. Schulte, J. M. Korn, R. A. Young, E. S. Lander, and N. Hacohen. 2001. The plasticity of dendritic cell responses to pathogens and their components. Science 294:870-875.
23. Silicon Genetics Inc. 2002. Class prediction.
24. Bammler T, Beyer R P, Bhattacharya S, et al. Standardizing global gene expression analysis between laboratories and across platforms. Nat. Methods. 2005; 2:351-356.
25. Irizarry R A, Warren D, Spencer F, et al. Multiple-laboratory comparison of microarray platforms. Nat. Methods. 2005; 2:345-350.
26. Larkin J E, Frank B C, Gavras H, Sultana R, Quackenbush J. Independence and reproducibility across microarray platforms. Nat. Methods. 2005; 2:337-344.
27. Jenner R G, Young R A. Insights into host responses against pathogens from transcriptional profiling. Nat Rev Microbiol. 2005; 3:281-294.
28. Pai R K, Pennini M E, Tobian A A, Canaday D H, Boom W H, Harding C V. Prolonged toll-like receptor signaling by Mycobacterium tuberculosis and its 19-kilodalton lipoprotein inhibits gamma interferon-induced regulation of selected genes in macrophages. Infect Immun. 2004; 72:6603-6614.

What is claimed is:

1. A method of identifying a human subject suspected of having an infectious disease comprising determining the expression level of a biomarker comprising one or more of the following genes: cig5; DNAPTP6; IFI27; IFI35; IFI44; OAS1; BST2; G1P2; LY6E; MX1; SON; TRIM14; APOBEC3C; Clorf29; FLJ20035; FLJ38348; HSXIAPAF1; KIAA0152; PHACTR2; and USP18.

2. The method of claim 1, wherein the step of determining expression levels is performed by measuring amounts of mRNA, protein and combinations thereof.

3. The method of claim 1, wherein the step of determining expression levels is performed using hybridization of nucleic acids on a solid support, an oligonucleotide array, sequencing and combinations thereof.

4. The method of claim 1, wherein the step of determining expression levels is performed using cDNA which is made using mRNA collected from the human cells as a template.

5. The method of claim 1, wherein the biomarker comprises mRNA level and is quantitated by a method selected from the group consisting of polymerase chain reaction, real time polymerase chain reaction, reverse transcriptase polymerase chain reaction, hybridization, probe hybridization, and gene expression array.

6. The method of claim 1, wherein the step of determining the level of expression is accomplished using at least one technique selected from the group consisting of polymerase chain reaction, heteroduplex analysis, single stand conformational polymorphism analysis, ligase chain reaction, comparative genome hybridization, Southern blotting, Northern blotting, Western blotting, enzyme-linked immunosorbent assay, fluorescent resonance energy-transfer and sequencing.

7. The method of claim 1, wherein the sample comprises a peripheral blood mononuclear cell.

8. A method of identifying a human subject suspected of having an infectious disease comprising determining the expression level of a biomarker comprising one or more of the following genes: EEF1G; EIF3S5; EIF3S7; EIF4B; QARS; RPL31; RPL4; PFDN5; CD44; HADHA; PCBP2; and dJ507I15.1.

9. The method of claim 1, wherein the step of determining expression levels is performed by measuring amounts of mRNA, protein and combinations thereof.

10. The method of claim 1, wherein the step of determining expression levels is performed using hybridization of nucleic acids on a solid support, an oligonucleotide array, sequencing and combinations thereof.

11. The method of claim 1, wherein the step of determining expression levels is performed using cDNA which is made using mRNA collected from the human cells as a template.

12. The method of claim 1, wherein the biomarker comprises mRNA level and is quantitated by a method selected from the group consisting of polymerase chain reaction, real time polymerase chain reaction, reverse transcriptase polymerase chain reaction, hybridization, probe hybridization, and gene expression array.

13. The method of claim 1, wherein the step of determining the level of expression is accomplished using at least one technique selected from the group consisting of polymerase chain reaction, heteroduplex analysis, single stand conformational polymorphism analysis, ligase chain reaction, comparative genome hybridization, Southern blotting, Northern blotting, Western blotting, enzyme-linked immunosorbent assay, fluorescent resonance energy-transfer and sequencing.

14. The method of claim 1, wherein the sample comprises a peripheral blood mononuclear cell.

15. A method of identifying a human subject suspected of having a infectious disease comprising differentiating between an infection with S. aureus infection and an E. coli infection by determining the expression level of a biomarker comprising one or more of the following genes: CXCL1; JAG1; RGS2; GAPD; PPIB; PSMA7; MMP9; p44S10; TRAM2; SEC24C; ACTG1; CGI-96; MGC2963; and STAU.

16. The method of claim 15, wherein the step of determining expression levels is used to detect E. coli infection as compared with S. aureus by determining the expression level of a biomarker comprising one or more of the following genes: RASA1; SNX4; AF1Q; SMAD2; JUP; PP; MAN1C1; FLJ10287; FLJ20152; LRRN3; SGPP1; and UBAP2L.

17. The method of claim 1, wherein the step of determining expression levels is performed by measuring amounts of mRNA, protein and combinations thereof.

18. The method of claim 1, wherein the step of determining expression levels is performed using hybridization of nucleic acids on a solid support, an oligonucleotide array, sequencing and combinations thereof.

19. The method of claim 1, wherein the step of determining expression levels is performed using cDNA which is made using mRNA collected from the human cells as a template.

20. The method of claim 1, wherein the biomarker comprises mRNA level and is quantitated by a method selected from the group consisting of polymerase chain reaction, real time polymerase chain reaction, reverse transcriptase polymerase chain reaction, hybridization, probe hybridization, and gene expression array.

21. The method of claim 1, wherein the step of determining the level of expression is accomplished using at least one technique selected from the group consisting of polymerase chain reaction, heteroduplex analysis, single stand conformational polymorphism analysis, ligase chain reaction, comparative genome hybridization, Southern blotting, Northern blotting, Western blotting, enzyme-linked immunosorbent assay, fluorescent resonance energy-transfer and sequencing.

22. The method of claim 1, wherein the sample comprises a peripheral blood mononuclear cell.

23. A method of identifying a human subject suspected of having an infectious disease by determining the expression level of a biomarker having one or more of the following genes to differentiate between a bacterial versus a viral infection: EEF1G; EIF3S5; EIF3S7; EIF4B; QARS; RPL31; RPL4; PFDN5; CD44; HADHA; PCBP2; and dJ507I15.1.

24. A computer implemented method for determining the phenotype of a sample comprising:
obtaining one or more probe intensities from a sample;
diagnosing an infectious disease based upon the probe intensities;
calculating linear correlation coefficient between the probe intensities and reference probe intensities; and
accepting the tentative phenotype as the genotype of the sample if the linear correlation coefficient is greater than a threshold value.

25. A computer readable medium comprising computer-executable instructions for performing the method for determining the transcriptome of a sample comprising:
obtaining a plurality of sample probe intensities;
diagnosing an infectious disease based upon the sample probe intensities for six or more genes selected those genes listed in Table 2, Table 3, Supplementary Tables 1 to 11 and combinations thereof; and
calculating a linear correlation coefficient between the sample probe intensities and reference probe intensities; and accepting the tentative genotype as the genotype of the sample if the linear correlation coefficient is greater than a threshold value.

26. The system of claim 25, wherein the biomarkers are selected from 5, 6, 7, 8, 9, 10, 11, 12 or more genes.

27. The system of claim 25, wherein the biomarkers are selected from one or more genes listed in Supplementary Tables 1 to 11, and combinations thereof.

28. A computer-based method for creating a datasets that correlates to the presence of an infectious disease in an individual, the method comprising computer-implemented steps of:
obtaining a plurality of gene probe intensities from the individual;
determining the probe intensities for six or more genes selected those genes listed in Table 2, Table 3, Supplementary Tables 1 to 11 and combinations thereof; and
calculating a linear correlation coefficient between the sample probe intensities and a reference probe intensity for each of the six or more genes, wherein the correlations are averaged across the six or more genes to calculate a transcriptome expression vector that correlates with the presence or absence of the infectious disease.
